(19) United States
(12) Patent Application Publication
(10) Pub. No.: US 2010/0310471 A1
(43) Pub. Date: Dec. 9, 2010

(54) ANTIOXIDANT AND PARAMAGNETIC HEPARIN-NITROXIDE DERIVATIVES

(75) Inventors: Andrey Kleschyov, Mainz (DE); Thomas Munzel, Mainz (DE); Valery Golubev, Chemogolovka (RU); Vasily Sen, Chemogolovka (RU)

Correspondence Address: CLARK & ELBING LLP, 101 FEDERAL STREET, BOSTON, MA, 02110, US

(73) Assignee: Johannes Gutenberg-Universität Mainz, Mainz (DE)

(21) Appl. No.: 12/675,583

(22) PCT Filed: Aug. 22, 2008
(86) PCT No.: PCT/EP08/06901

§ 371 Date: May 26, 2010

(30) Foreign Application Priority Data
Aug. 28, 2007 (EP) 07 016 789.5

(51) Int. Cl. A61K 49/12; C08B 37/10; A61K 31/727; A61P 9/00; A61P 25/28; A61P 29/00; A61P 35/00; A61P 3/10; A61P 9/12; A61P 9/10; A61P 19/02; A61B 5/055

(52) U.S. Cl. 424/9.33; 536/21; 514/56; 600/420

(57) ABSTRACT

The present invention relates to novel heparin-nitroxide derivatives comprising heparin and at least two and more nitroxides/polynitroxide radicals that are covalently coupled to heparin by derivatisation of glycosaminoglycan carboxyl or amino groups. The heparin-nitroxide derivatives are useful as therapeutic or diagnostic agents. This invention further concerns novel methods for the production of the heparin-nitroxide agents, and methods of their uses for specifically targeting and labelling of biological vessels. The inventions also suggest the uses of the heparin-nitroxide derivatives for treatment of oxidative stress-mediated diseases. Furthermore, the heparin-nitroxide derivatives according to the present invention are in particular useful for electron paramagnetic resonance imaging (EPRI), for magnetic resonance imaging (MRI), and for preservation of biological transplants.

### TECHNICAL FIELD

The present invention relates to novel therapeutic and diagnostic agents comprising heparin and at least two and more nitroxides or polynitroxides that are covalently coupled to heparin by derivatisation of glycosaminoglycan carboxyl or amino groups.

The polynitroxide-heparin derivatives of the invention are useful as therapeutic agent or diagnostic probe.

In another aspect, the invention concerns novel methods for the production of the heparin-nitroxide derivatives, and methods of their uses for specifically targeting and labelling of blood vessels.

The inventions also suggest the uses of the polynitroxide-heparin derivatives for treatment of extracellular oxidative stress-mediated diseases.

In another aspect, the heparin-nitroxide derivatives according to the present invention can be in particular useful for electron paramagnetic resonance imaging (EPRI), for magnetic resonance imaging (MRI), and for preservation of biological transplants.

### BACKGROUND ART

Overproduction of oxygen-derived free radicals/reactive oxygen species (ROS) and the effect of these toxic molecules on cell function are collectively called “oxidative stress.” Oxidative stress results from an imbalance between the formation and neutralization of ROS within cells and/or extracellular space. For instance, ROS cause oxidative stress in endothelial cells, a condition implicated in the pathogenesis of many cardiovascular and pulmonary diseases, and diabetes.

Such ROS like superoxide anion radical (O₂⁻.) and H₂O₂, are formed during a variety of biochemical reactions and cellular functions. The steady-state formation of these free radicals is normally balanced by a similar rate of their consumption by antioxidants. Relatively stable O₂⁻. and H₂O₂ can generate the highly reactive ROS, such as OH. radical, thiyl radicals and peroxynitrite which can react with various cellular components including DNA, proteins, lipids/fatty acids and accelerate formation of advanced glycation end products (e.g. carbonyls) and quench a key vascular mediator, nitric oxide. These reactions lead to the disruption of cellular compartments and/or function, e.g. DNA damage, mitochondrial malfunction, cell membrane damage and eventually cell death.

The fine control of ROS levels in the specific cell/tissue compartments (e.g. mitochondrium, cytosol, extracellular space) is crucial for the normal physiology. Under several pathophysiological conditions, the ROS levels can be sharply elevated in one, but not in another cell/tissue compartment. For example, the extracellular oxidative stress is important in pathological conditions such as ischemia/reperfusion, atherosclerosis and diabetes etc. Therefore, the development of specifically targeted ROS sensors and/or ROS scavenging agents would be very important, both for the early diagnosis and the efficient treatment of these disease states.

In blood vessels, the endothelium is a major site of both ROS production and ROS-induced injury. In one approach, scavenging of ROS at the endothelium is achieved by infusion of antioxidant enzymes, such as superoxide dismutases (SOD) and catalase (Beckman J S et al., J Free Radic Biol Med, 1986, 2(5-6):359-65). However, these enzymes are known to undergo a fast elimination from the bloodstream and permits rather modest, if any, protection against vascular oxidative stress.

It has therefore been proposed to couple polyethylene glycol (PEG) to the enzymes or encapsulate them in liposomes to increase bioavailability and to enhance the protective effect of SOD and catalase (Muzykantov V R, J Control Release, 2001, Mar. 12;71(1):1-21). However, the problem still remains with this approach to specifically scavenge free oxygen radicals at extracellular structures.

An further approach proposed chimeric protein constructs consisting of SOD and heparin-binding particles having an affinity for charged components of the endothelial glycocalix, or SOD/catalase conjugated antibodies directed against the constitutively expressed endothelial antigens, angiotensin-converting enzyme (ACE), and adhesion molecules (ICAM-1 or PECAM-1). However, none of these approaches led to a persistent and effective protection of endothelial cells and extracellular structures. Additionally, increasing SOD concentrations in tissues produce a paradoxical pro-oxidant effect (Nelson S K, Bose S K, McCord J M, Free Radic Biol Med, 1994, 16:195-200), which is in part due to the SOD-derived H₂O₂ and Cu²⁺, which cause the generation of highly reactive OH. radical.

Another class of compounds that have been shown to exhibit antioxidant activity and tissue protection are nitroxides (S. M. Hahn, F. J. Sullivan, A. M. DeLuca, J. D. Becher, J. Liebmann, M. C. Krishna, D. Coffin, J. B. Mitchel, “Hemodynamic effect of the nitroxide superoxide dismutase mimics”, Free Rad. Biol. Med., 1999, 27, 529-535; S. Zhang, H. Li, L. Ma, C. E. Trimble, P. Kappusamy, C. J. C. Hsia, D. L. Carden, “Polynitroxyl-albumin plus tempol attenuate lung capillary leak elicited by prolonged intestinal ischemia and reperfusion”, Free Rad. Biol. Med., 2000, 29, 42-50; R. Rak, D. L. Chao, R. M. Pluta, J. B. Mitchel, E. H. Oldfield, J. C. Watson., “Neuroprotection by the stable nitroxide tempol during reperfusion in a fat model of transient focal ischemia”, J. Nuerosurgery, 2000, 92, 646-651; K. Takeshita, K. Saito1, J. Ueda, K. Anzai, T. Ozawa, “Kinetic study on ESR signal decay of nitroxyl radicals, potent redox probes for in vivo ESR spectroscopy, caused by reactive oxygen species”, BBA, 2002, 1573, 156-164; T. Nassar, B. Kadery, N. Da'as, Y. Kleinman, A. Haj-Yahia, “Effects of the superoxide dismutase-mimetic compound tempol on endothelial disfunction in streptozotocin-induced diabetic rats”, Eur. J. Pharmacol., 2002, 436, 111-118; J. B. Mitchel, A. Samuni, W. G. DeGraff, S. Hahn, “Nitroxides as protectors against oxidative stress”, U.S. Pat. No. 6,605,619, 8/2003; W.-J. Liaw, T.-H. Chen, Z.-Z. Lai, S.-J. Chen, A. Chen, C. Tzao, J.-Y. Wu, C.-C. Wu, “Effects of a membrane-permeable radical scavenger, tempol, on intraperitoneal sepsis-induced organ injury in rats”, Shock, 2005, 23, 88-96).

Nitroxides (stable nitroxyl radicals, NR) have been used to protect cells against oxidative damage following cardiac arrest, brain, trauma, ischemia/reperfusion, and radiation (K. Patel, Y. Chen, K. Dennehy, J. Blau, S. Connors, M. Mendonca, M. Tarpey, M. Krishna, J. B. Mitchell, W. J. Welch, C. S. Wilcox, “Acute antihypertensive action of nitroxides in the spontaneously hypertensive rat”, Am. J. Physiol. Regulatory Integrative Comp. Physiol., 2006, 290, 37-43; F. Hyodo, K. Matsumoto, A. Matsumoto, J. B. Mitchell, M. C. Krishna, “Probing the intracellular redox status of tumors with magnetic resonance imaging and redox-sensitive contrast agents”, Cancer Research, 2006, 66, 9921-9928).

Nitroxides are known SOD mimetics, i.e they efficiently catalize the dismutation of O₂⁻. into H₂O₂ and O₂ (V. D. Sen', V. A. Golubev, I. V. Kulyk, E. G. Rozantsev, “Mechnism of the reaction of hydrogen peroxide with oxopiperidinium salts and piperidinoxyl radicals”, Rus. Chem. Bull., 1976, 25, 1647-1654; S. Goldstein, G. Merenyi, A. Russo, A. Samuni, “The role of oxoammonium cation in the SOD-mimic activity of cyclic nitroxides”, J. Am. Chem. Soc., 2003, 125, 789-795).

The SOD-mimetic activity of nitroxides is illustrated as follows:

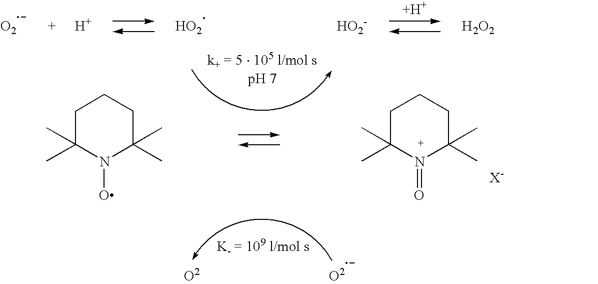

Additionally, nitroxides scavenge other ROS, such as H₂O₂, OH., thiyl radicals etc. As antioxidants, they undergo reversible reduction and oxidation to hydroxylamines (HAs) and oxoammonium cations (OCs) correspondingly (V. A. Golubev, Y. N. Kozlov, A. N. Petrov, A. P. Purmal, “Catalysis of redox processes by nitroxyl radicals”, in “Bioactive spin labels”, R. I. Zhdanov, ed., Springer, Berlin, 1992, pp. 119-140). The following scheme exemplifies the function of nitroxides in scavenging free radicals:

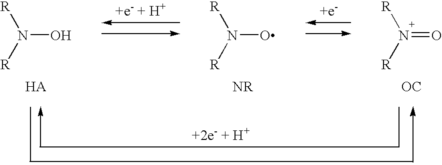

Among nitroxides, the cyclic nitroxides have been proven to exhibit unique antioxidant properties (Goldstein J. Am. Chem. Soc., 2003, (125), 789-795). Cyclic nitroxides are stablized by adding methyl groups at the alpha position in five-membered pyrrolidone, pyrroline or oxazolidine, and six-membered piperidine ring structures. The methyl groups confer stability to the nitroxide radicals by preventing radical-radical dismutations.

The cyclic nitroxide, TEMPO having the chemical structure of 2,2,6,6-tetramethylpiperidine-1-oxyl and its derivatives, for example, TEMPOL having the chemical structure of 4-hydroxy-2,2,6,6-tetramethylpiperidine-N-oxyl have been shown to be effective agents for potentially treating a number of oxidative stress-induced diseases. For instance, TEMPOL has been described as potential agent for treating neoplastic diseases, such as cancer (WO 2006/084197).

Since cyclic nitroxides are relatively stable free radicals, they are widely used as EPR-probes in biophysical studies (McConnel H M, Spin Labeling: Theory and Applications, 1976, New York: Academic Press). As such they have been utilized as redox-sensitive paramagnetic contrast agent in Magnetic Resonance Imaging (MRI) (Matsumoto; Clin Cancer Res, 2006, 12: 2455-2462) and for EPR imaging (EPRI) (Herrling T, Free Radic Biol Med, 2003, Jul. 1;35(1):59-67).

However, although cyclic nitroxides are promising agents that can be used both for the tissue antioxidant defense as well as for MRI and EPRI, the problem persists that they are free flowing and easily eliminated from the bloodstream in vivo. As a further problem, conventional nitroxides must be used at high (mM) concentrations to be effective since they enter cells and undergo a rapid reduction process. In addition, intracellular nitroxides may negatively interfere with the cellular metabolism. Therefore, it appears to be desirable to have an agent, which would allow the targeting of nitroxides to specific compartment of biological tissue.

Recently, the mitochondria-targeted nitroxide (Mito-TEMPOL) has been proposed to selectively inhibit the mitochondrial oxidative damage (reviewed in: Murphy M P and Smith R A J, Annual Review of Pharmacology and Toxicology, 2007, 47:629-656). However, pharmacological tools for specific targeting of nitroxides to another strategically important sites, such as endothelial cell surface and extracellular matrix (ECM) are still lacking.

The extracellular matrix consists of a complex mixture of proteins and glycoproteins (e.g. fibrilin) serving multiple functions such as in cellular growth development, angiogenesis and tissue regeneration. The extracellular matrix is critical for all aspects of vascular biology. In concert with supporting cells, endothelial cells assemble a laminin-rich basement membrane matrix that provides structural and organizational stability. Expression and activity of the key ECM enzyme, matrix metalloproteinase(s), which is largely responsible for the atherosclerotic plaque instability, is extremely sensitive to oxidative stress. Atherosclerosis, ischemia-reperfusion and vascular inflammation are known to be associated with the recruitment of activated macrophages and polynuclear leucocytes causing extracellular oxidative stress.

In the prior art, the cyclic nitroxide (e.g. TEMPO) coupled to a polymer dextran have been described (Kazama S. et al., R, Resonance in Medicine, 1996, 36: 547-550; Saito K et al., Bioscience Biotechnology and Biochemistry, 65: 787-794; US-B1-6458758). However, dextran-nitroxides are not suitable for the specific recruitment of nitroxides to the endothelial cells and vascular ECM.

Dextran is a neutral polymer, consisting of 1→6 bonded α-D-glucopyranose monomers.

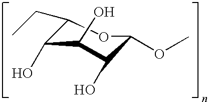

There is another widely clinically used polysaccharide, namely heparin. Heparin is a member of the glycosaminoglycan family of carbohydrates (which includes the closely related molecule heparan sulfate) and composed of variably sulfated repeating disaccharide units (D. S. Milbrath, R. H. Ferber, W. E. Barnett, “Diagnostic radio-labeled polysaccharide derivatives”, U.S. Pat. No. 4,385,046, 5/1983). The main disaccharide unit of heparin is 2-O-sulfo-L-idopyranosyluronic acid (1→4) linked to 2-N-6-O-disulfo D-glucosamine. The average heparin disaccharide contains 2.7 sulfo groups (I. Capita, R. J. Linhardt, “Heparin-protein interactions”, Angew. Chem. Int. Ed., 2002, 41, 390-412; R. J. Linhardt, “Heparin: structure and activity”, J. Med. Chem., 2003, 46, 2551-2564).

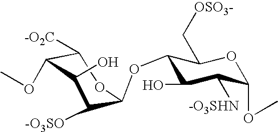

Glycosaminoglycans are highly negatively charged polysaccharides (having specific distribution of the charge within macromolecule) and due to this property are able to bind to the specific positively charged sites on the cell surface and extracellular matrix of biological tissues. By contrast, dextran-nitroxide derivatives can fill the accessible biological compartments and associate non-specifically and weakly with tissue structures

Rota C et al. had reported the formation of nitroxide-heparin fragments during the chemically-induced depolymerization of heparin macromolecule (Rota C et al., Res Chem Intermed; Research on Chemical Intermediates, 2006, 32: 73-81). This document, however, does not deal with derivatised heparin-nitroxides conjugates and does not imply any cardiovascular applications for these derivatives. Rather the study investigated the mechanism of the process when un-fractionated heparin is converted into the low molecular weight heparin by adding the copper salt and hydrogen peroxide. The results suggest that during the reaction of depolymerization, some unidentified nitroxide radicals can be formed (among other radicals). It has been suggested in this paper that hydroxyl radicals generated in the copper/hydrogen peroxide system may directly attack and oxidize some amino groups present in heparin into unstable nitroxide radicals of unknown nature. The nitroxide-containing product of this reaction is chemically different from the polynitroxide-heparin derivative of the invention and does not relate to the problems underlying the present invention.

Gemma E et al. describe a DMT-MM mediated functionalisation of the non-reducing end of disaccharide unit of glycosaminoglycans (Gemma E et al., Chemical Communications, 2007, 26: 2686-2688). In this study, only disaccharides obtained by glycosaminoglycan depolymerization, were labelled with TEMPO. In contrast to polymeric glycosaminoglycans, this low molecular weight derivative cannot form net charge stabile binding on the cell surface and extracellular matrix of biological tissues and, therefore, does not relate to the problems underlying the present invention. The inventors surprisingly discovered the antioxidant and paramagnetic properties for this product, which were not evident or expected. It was also surprising that these products can be used for nitroxide radical detection (EPR).

US 2006/014720 deals with heparin-prodrugs, which implies that the active ingredient, (the actual drug) will be released from the pharmacologically inert heparin-conjugated form upon its biodegradation in tissues. In this embodiment, the hydrolytically (or enzymatically) unstable linkage between heparin and drug is essential. The authors proposed to use the following types of heparin-based prodrugs: 1) ester-type prodrug: bound between carboxyl group of heparin and hydroxyl group of drug or vice versa. 2) Schiff-base-type prodrug: aminogroup of drug and heparin functionalized to have aldehyde group or vice versa. 3) acetal- or hemi-acetal-type prodrug: hydroxyl groups on drug and heparin functionalized to have aldehyde group or vice versa. In contrast, as will be shown, the present invention requires a stable linkage between heparin and nitroxide.

The heparin-prodrug composition described in US 2006/014720 is used for coating of a medical device being inserted or implanted to human beings (e.g. stents, catheters etc), but not for infusion into the blood stream. The described embodiment does not imply that heparin-prodrug derivative will be quenched by the specific heparin-binding sites in blood vessels to provide both the therapeutic effect and diagnostic information.

### TECHNICAL PROBLEM

It is the object of the present invention to provide a therapeutically and diagnostically effective agent, which allows for the selective delivery and stable association of nitroxides with endothelial cells and extracellular structures, for labelling of blood vessels for magnetic resonance imaging, or for protecting biological vessel against oxidative stress.

### SOLUTION OF THE TECHNICAL PROBLEM

The above problem is solved by a heparin-nitroxide derivative comprising the technical features as defined in claim 1.

### DESCRIPTION OF THE INVENTION

The polynitroxide-heparin derivatives of the invention comprise cyclic nitroxides conjugated with the heparin/glycoaminoglycan backbone at multiple sites, preferably via amide bounds. The products are able to bind with high affinity to the heparin-binding-sites on endothelial cell surface and vascular extracellular matrix (ECM), thereby exhibiting a prolonged bioavailability.

The terms “heparin” as used in the context of the present invention comprises native heparin, both fractionated and unfractioned (UH) heparin preparations, and low molecular weight heparin (LMWH). Also encompassed by the present invention are mixtures of heparin preparations with either varying molecular weights or specific molecular weights, and the closely related molecule of heparan sulfate.

A “derivative” as used for the present invention defines a compound that is formed from a similar compound or constitutes a modified compound. A derivative may be obtained, for instance, by replacing one atom with another atom or group of atoms. The term encompasses any modification, variant, or analogue of a compound.

A “heparin-nitroxide derivative” or “polynitroxide-heparin derivative” according to the invention is meant to be heparin (or the highly similar heparan sulfate) conjugated with multiple (preferably cyclic) nitroxides. The nitroxides are preferably conjugated via amide bounds. In this application, the conjugation product of heparin and nitroxides is referred to as polynitroxide-heparin or heparin-nitroxide.

The terms “nitroxide” and “nitroxyl radical” essentially refer to the same compound and are equally used in the context of the present application.

The term “polynitroxide-heparin” is meant to comprise nitroxides bound to heparin at the carboxyl or amino group of the glycoaminoglycan backbone (preferably via amide linkage).

Preferably, more than 20% of disaccharides of the heparin glycoaminoglycan macromolecule are bound by nitroxides/nitroxyl radicals. In other embodiments more than 20%, 30%, 40%, 50%, 60%, 70% of disaccharides of the heparin glycoaminoglycan macromolecule are labelled by nitroxides/nitroxyl radicals. In a further embodiment more than 90% of disaccharides of the heparin glycoaminoglycan macromolecule are labelled by nitroxides/nitroxyl radicals.

A number of heparin-binding sites have been identified in the constituents of the ECM, e.g. in fibrilin and collagen, the later being a major structural protein of ECM. The antioxidant heparin-nitroxide constructs of the invention allow for directed targeting of nitroxides to the extracellular compartments. By binding to the heparin-binding sites at cell surfaces and ECM, they are able to exhibit their beneficial effects, i.e. the prevention or diminishing of oxidative stress on the cell surface and in ECM. This is particularly important to inhibit oxidative stress in the intima layer of blood vessels. Importantly, the rapid entry of nitroxide moieties into cells and interference with intracellular metabolism is avoided.

As such the heparin-nitroxide derivatives of the invention provide useful therapeutic tools for treating and preventing vascular diseases such as, for instance, ischemia/reperfusion, atherosclerosis, inflammation or diabetes.

In addition to the above-mentioned therapeutic benefits, selective targeting of nitroxides to extracellular compartments potentially allows for diagnostic purposes such as EPRI and MRI of the vascular structure of vessels or monitoring of local vascular oxidative stress in vivo. Due to lack of interaction with intracellular reducing agents and prolong half-life time of nitroxides, heparin-nitroxides of the invention can be in particular useful for in vivo EPRI and MRI. As such, heparin being conjugated with the particular nitroxides can be used as redox, pH and dioxygen sensors in the specific tissue compartment.

Furthermore, the antioxidant heparin-nitroxide constructs of the invention can prevent oxidative stress-dependent platelet activation by scavenging of reactive oxygen species and preservation of endogenous NO activity. Thus, heparin-nitroxide of the invention can improve the anticoagulant properties of such widely used drug as heparin.

Heparin has the highest negative charge density of any known biological molecule and, as a result, binds efficiently to positively charged biological targets such as extracellular structures like ECM.

Native heparin is a polymer with a molecular weight ranging from 3 kDa to 50 kDa although the average molecular weight of most commercial heparin preparations is in the range of 12 kDa to 18 kDa. In its natural unfractionated state, heparin exists as a heterogeneous mixture of oligosaccharides composed of alternating chains of D-glucosamine and uronic acid.

Possible heparin modifications that may be used for the construction of the agents of the invention are described, for example, in J. Gang, J. Wang, “Use of N-desulfated heparin for treating or prevention of inflammation”, EP1300153, 4/2003; P. E. Thorpe, “Preparation and use of steroid-polyanionic polymer-based conjugates targeted to vascular endothelial cells”, U.S. Pat. No. 5,474,765, 12/1995; P. E. Thorpe, “Method of using of steroid-polyanionic polymer-based conjugates targeted to vascular endothelial cells”, U.S. Pat. No. 5,762,918, 6/1998; Y. Byun, Y.-K. Lee, “Oral delivery of macromolecules”, US2002/0010153, 1/2002; A. D. Cardin, C. L. Van Gorp, “Targeted agents useful for diagnostic and therapeutic applications”, U.S. Pat. No. 6,409,987, 6/2002.

Preferably the heparin or derivative thereof as utilized in the heparin-nitroxide derivative of the invention has a molecular weight of approximately between 5 and 40 kDa. Most preferred is a molecular weight of around 15 kDa of fractionated heparin. The following schemes illustrate possible ways of a conjugation of heparin with nitroxide (R) via an optional linker (L). By adding a linker chain L, the nitroxide molecule will be placed in greater distance from the heparin macromolecule, which results in a higher spectral mobility as further shown in the Examples. The amide bound (also known as peptide bound), —C(O)NH—, is preferably used for the synthesis of the polynitroxide-heparin of the invention. The in vivo stability of the polynitroxide-heparin for which the linker is crucial is supported by the inventors' data.

The basic structure of the heparin macromolecule is shown in scheme 1a, possible heparin-nitroxides conjugations are shown in schemes 1b and 1c, respectively.

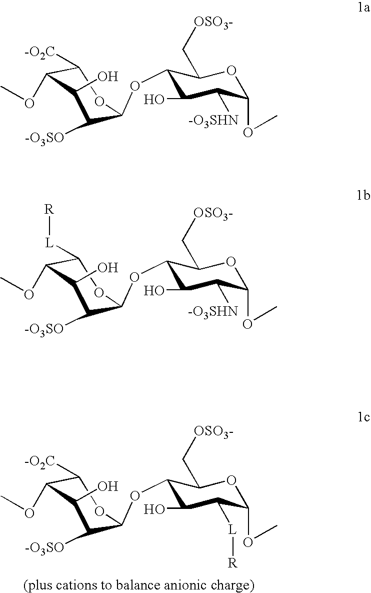

Preferred nitroxides or their derivatives according to the invention are derived from cyclic nitroxides. A preferred nitroxide to be utilized in the present invention is TEMPO (2,2,6,6-tetramethylpiperidine-1-oxyl). Preferred nitroxides or derivatives thereof are derived from piperidine, tetrahydropyridine, pyrroline, pyrrolidine, imidazoline, imidazolidine or oxazolidine.

One advantage of the agent according to the present invention is that a large number of nitroxyl radicals can be coupled to the heparin macromolecule. The nitroxide compounds can be the same or different. Accordingly, by coupling a sufficiently high number of nitroxyl radicals to the glycoaminoglycan backbone of heparin, the antioxidant and paramagnetic properties are highly increased as compared to compounds in which only a minor number such as one or two of molecules are coupled to a glycoaminoglycan carrier where no or only minor effects are detectable.

In comparison to known dextran-nitroxide of the prior art, the heparin-nitroxide derivatives of the invention are able to bind with high affinity to specific sites of biological tissue. These specific heparin-binding sites expressed in biological tissues are of exceptional physiological importance. Many redox-sensitive receptors, growth factors, cytokines, as well as various pro-oxidant (e.g. myeloperoxidase) and anti-oxidant enzymes (e.g. extracellular form of superoxide dismutase) are bound to these particular sites. Therefore, the precise targeting of the antioxidant nitroxides to this important location appears to be a very promising therapeutic strategy. Additionally, the heparin-nitroxide agent of the invention can be considered as an valuable molecular probe for labelling and MR probing of the redox state at specific sites of the vascular wall. Clearly, any of the known dextran-nitroxide derivatives cannot be used for these purposes.

The inventors will demonstrate in the following examples that dextran-nitroxide compounds are not able to bind to the specific heparin-binding sites in vascular tissue (see Experiment 9 below).

Preferably, more than 50%, even more preferred more than 70% of the possible carboxyl and/or amino group binding sites at the glycoaminoglycan backbone of heparin are occupied by nitroxide compounds. In one embodiment all or almost all binding sites of heparin are occupied with nitroxides. The more nitroxyl radicals are bound to heparin, the better are the antioxidant and paramagnetic properties of the agent according to the invention. For some applications, however, it can be sufficient that only 20% of the binding sites of heparin are occupied with nitroxide. Occupation of heparin with only one nitroxide is not considered to be sufficient for antioxidant defense.

In view of the potential uses of the heparin-nitroxide derivatives of the invention, the invention also comprises an antioxidant agent and a paramagnetic probe for therapeutic and diagnostic purposes, respectively.

### Methods of Making Antioxidant Heparin-Nitroxide Constructs of the Invention

Nitroxide or its derivative can be linked to the glycosaminoglycan backbone of heparin by derivatisation of glycosaminoglycan reactive groups. Preferred reactive groups for coupling nitroxide to heparin are —COOH or —NH₂.

Although any of known coupling reactions may be used for making the heparin-nitroxide derivatives of the invention, there are two preferred ways of derivatisation glycosaminoglycan reactive groups of heparin:

1. Derivatisation of heparin carboxyl groups with amino group containing nitroxides, or

2. Derivatisation of heparin amino groups with carboxyl group containing nitroxides.

Any of the —COOH or —NH₂ (after desulfation) groups of the heparin macromolecule are suitable for derivatisation with nitroxides. However, poor solubility of heparin in solvents other then water or water-DMF (DMSO) mixtures may eventually limit the options for reagents options and types of activation reaction for derivatisation. Soluble in non-polar organic solvents heparin salts associated with bulky ammonium cations are preferred when the derivatisation reagents, such as acid chlorides, require anhydrous reaction conditions.

1. Derivatisation of Heparin Carboxyl Groups

One preferred method of making the heparin-nitroxide derivatives of the invention comprises carbodiimide-mediated coupling of heparin carboxyl groups with amino group containing substances of interest (see formula 1b, above). This reaction results in heparin derivatives having the following general formula, wherein the nitroxide R is a 5- or 6-atom N-heterocycle, L is a linker which comprises amide —C(O)NR′—, or diamide separated by hydrocarbon chain —C(O)NR′—(CH₂)ₘ—C(O)NR′— (CH₂)ₙ— or —C(O)NR′—(CH₂)ₘ—NR′C(O)—(CH₂)ₙ— or by any other suitable linker, R′ is a hydrogen or an alkyl substituent, m indicates the length of first hydrocarbon chain of the linker, wherein m≦0, preferably m is an integer >1, n indicates the length of second hydrocarbon chain of the linker, wherein n≦0, preferably n is an integer from 0 to 2, and x,y>0.

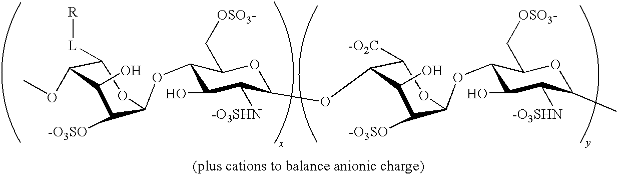

Any amino group containing nitroxide, including nitroxides with an additional heteroatom in the backbone cycle, like imidazoline, imidazolidine or oxazolidine, are suitable for the derivatisation of heparin according to the method of the invention.

Preferred nitroxides of the present invention are those with the highest k+limiting rate constant (see schema on page 4), i.e. nitroxides that are predominantely oxidizable by HO₂. radicals (HO₂. [symbol] O₂.—+H⁺).

Piperidine nitroxides are preferable as SOD-mimetics because the pyrrolidine/pyrroline nitroxide reaction with HO₂. is characterized by a lower k+limiting rate constant.

Imidazoline and imidazolidine types of nitroxides bound to heparin are preferable as extracellular pH-sensitive EPR probe.

Perdeuterated nitroxides, which are known to exhibit a very narrow EPR signal (0.08 gauss), are preferred for EPRI purpose and as oxymetry probe.

A preferred solvent for heparin is water. In a further embodiment, water-soluble carbodiimides are preferable such as, for instance, N-(3-dimethylaminopropyl) N′-ethylcarbodiimide hydrochloride (EDC).

Furthermore, the use of auxiliary N-hydroxy succinimide (NHS) can improve the efficiency of amide bond formation:

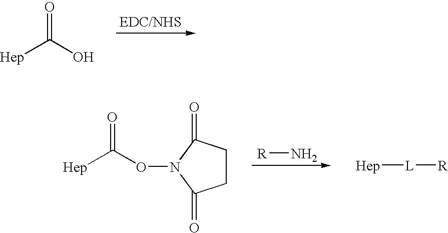

In this scheme, Hep-COOH refers to the heparin macromolecule with one of its carboxyl group, R—NH₂ refers to an amino group containing nitroxyl radical and L is a linker. Any suitable linker may be used for the invention that is able to conjugate nitroxide to the heparin macromolecule. The length of the linker may influence the antioxidant and paramagnetic properties of the bound nitroxyl radical. A longer linker chain, for instance, places the nitroxide molecule in greater distance from the heparin backbone, and thus may give different EPR signal properties. In fact, as shown in the Examples below, the signal width of the EPR spectrum (X- or L-band) may be influenced by the linker length.

The linker (L) preferably comprises amide bond —C(O)NR′— or diamide bonds separated by hydrocarbon chain —C(O)NR′—(CH₂)ₘ—C(O)NR′—(CH₂)ₘ— or —C(O)NR′— (CH₂)ₘ—NR′C(O)—(CH₂)ₙ— or by any other suitable linker, R′ is a hydrogen or an alkyl substituent, m indicates the length of first hydrocarbon chain of the linker, wherein m>0, preferably m is an integer >1, n indicates the length of second hydrocarbon chain of the linker, wherein n≦0, preferably n is an integer from 0 to 2.

Amino group containing nitroxides with flexible linkers of variable length may be obtained as shown for 4-[(5-aminopentyl)carbonylamino]-2,2,6,6-tetramethylpiperidine-1-oxyl (see also Example 1):

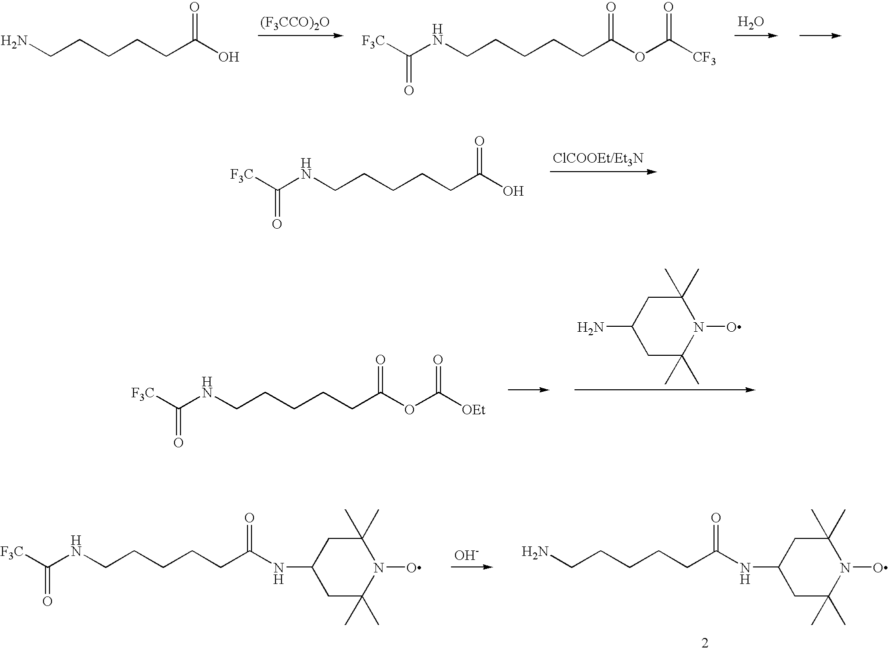

For the coupling reaction, the preferred NHS/R—NH₂ molar ratios are within the range from 1:10 to 1:1. In order to obtain higher degrees of derivatisation, carbodiimide is preferably added to the mixture of heparin/NHS/amino-nitroxide rather than the other way round. The degree of heparin derivatisation depends substantially on the temperature profile of the reaction. The best results are obtained when during the first 30 to 90 min the reaction mixture is kept in an ice bath and is then subsequently warmed up to a temperature of around 20° C. Depending on the relationship of the reagents and the duration of the reaction, the degree of heparin derivatisation may be changed up to one radical per disaccharide unit.

Preferred nitroxides as used in the present invention comprise HOOC(CH₂)n- or R′HN(CH₂)n-functionalyzed nitroxides, wherein n≦0, preferably n is an integer from 0 to 2, and R′ is H or Me.

In preferred heparin-nitroxide derivatives, the following nitroxide radical moieties R═R¹⁻¹¹ are suitable:
R1=2,2,6,6-tetramethyl-1-oxylpiperidin-4-yl,
R2=3-amino-2,2,6,6-tetramethyl-1-oxylpiperidin-4-yl,
R3=4-alkyloxycarbonyl-2,2,6,6-tetramethyl-1-oxylpiperidin-4-yl,
R4=4-hydroxyimino-2,2,6,6-tetramethyl-1-oxylpiperidin-3-yl,
R5=2,2,5,5-tetramethyl-1-oxylpyrrolidin-3-yl,
R6=2,2,6,6-tetramethyl-1-oxylpiperidin-4-diyl,
R7=2,2,5,5-tetramethyl-1-oxylpyrrolidin-3-diyl,
R8=2,2,6,6-tetramethyl-1-oxyl-1,2,5,6-tetrahydropyridin-4-yl,
R9=4-acetylamino-2,2,6,6-tetramethyl-1-oxylpiperidin-4-yl,
R10=2,2,5,5-tetramethyl-1-oxylpyrrolin-3-yl,
R11=2,2,5,5-tetramethyl-4-bromo-1-oxylpyrrolin-3-yl.

In the following, structures of antioxidant heparin-nitroxides agents of preferred embodiments are presented as obtained by coupling amino group containing nitroxide to carboxyl group containing heparin. For further details on their production please refer to the Examples.

One amide bond linkers (see Examples 2, 3):

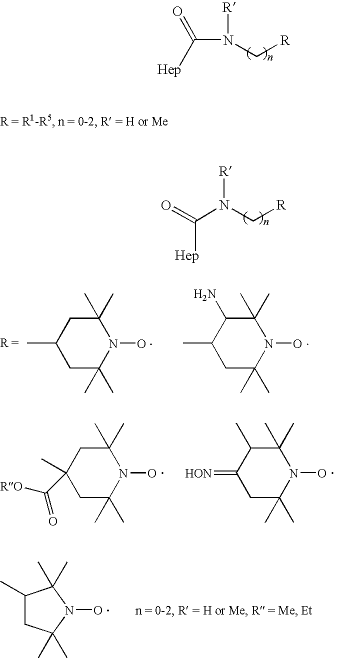

Hydrazide-hydrazone bond linker (see Examples 5-7):

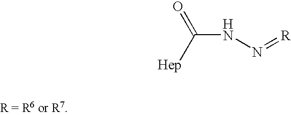

Two amide bonds linkers (see Example 4):

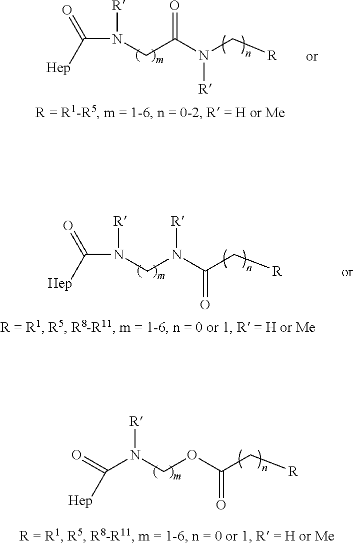

wherein R═R¹⁻¹¹ may be one of the following residues:
R¹=2,2,6,6-tetramethyl-1-oxylpiperidin-4-yl,
R²=3-amino-2,2,6,6-tetramethyl-1-oxylpiperidin-4-yl,
R³=4-alkyloxycarbonyl-2,2,6,6-tetramethyl-1-oxylpiperidin-4-yl,
R⁴=4-hydroxyimino-2,2,6,6-tetramethyl-1-oxylpiperidin-3-yl,
R⁵=2,2,5,5-tetramethyl-1-oxylpyrrolidin-3-yl,
R⁶=2,2,6,6-tetramethyl-1-oxylpiperidin-4-diyl,
R⁷=2,2,5,5-tetramethyl-1-oxylpyrrolidin-3-diyl,
R⁸=2,2,6,6-tetramethyl-1-oxyl-1,2,5,6-tetrahydropyridin-4-yl,
R⁹=4-acetylamino-2,2,6,6-tetramethyl-1-oxylpiperidin-4-yl,
R¹⁰=2,2,5,5-tetramethyl-1-oxylpyrrolin-3-yl,
R¹¹=2,2,5,5-tetramethyl-4-bromo-1-oxylpyrrolin-3-yl.

The number of molecules of nitroxides over the full length of the heparin macromolecule can vary. A preferred number of nitroxides per heparin macromolecule is, for instance, approximately 2 to 24 nitroxides per heparin macromolecule having a molecular weight of around 15 kDa. In a preferred embodiment, at least 20%, preferably about 20% to 70% of the disaccharides of heparin are modified by the cyclic nitroxide TEMPO. In further embodiments, at least 20%, 45% or 70% of the disaccharides of heparin are modified by TEMPO. In a yet preferred embodiment more than 70% of the disaccharides of heparin are modified by TEMPO.

### 2. Heparin Amino Groups Derivatisation

The second preferred method of making the antioxidant heparin-nitroxides of the invention comprises N-desulfation/N-acylation of heparin resulting in an heparin-nitroxide derivative of the following general formula:

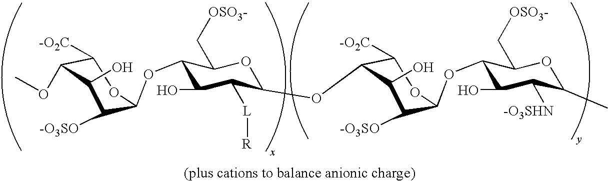

wherein the nitroxide R is a 5- or 6-atom N-heterocycle, L is an optional linker, and x,y>1. Preferably, the linker L comprises natural amino acids or short peptides residues, amide bond —NR′C(O)— or diamide bonds separated by hydrocarbon chain —NR′C(O)—(CH₂)ₘ—C(O)NR′—(CH₂)ₙ— or —NR′C(O)—(CH₂)ₘ—NR′C(O)—(CH₂)ₙ— or by any other suitable linker, R′ is a hydrogen or an alkyl substituent, m indicates the length of first hydrocarbon chain of the linker, wherein m>0, preferably m is an integer >1, n indicates the length of second hydrocarbon chain of the linker, wherein n≦0, preferably n is an integer from 0 to 2.

The derivatisation of heparin amino groups is preferably performed after N-desulfation of heparin. In a preferred method of the invention, NHS esters of carboxyl group containing nitroxides (NHS—C(O)R) were obtained and coupled to —NH₂ groups of N-desulfated heparin Hep-NH₂ in a water-DMSO mixture according to the following reaction scheme:

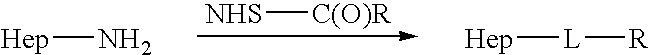

In this scheme, L represents a linker as defined under the above. In principle, any carboxyl group containing nitroxide is suitable for heparin derivatisation according to this variant of the method of the invention.

In the following, structures of antioxidant heparin-nitroxides agents are presented as obtained by coupling carboxyl group containing nitroxides or a derivative thereof to amino group containing heparin. For further details as regards to their production please refer to the Examples that follow under the below.

One amide bond linkers (see Example 8):

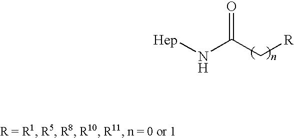

Two amide bond linkers:

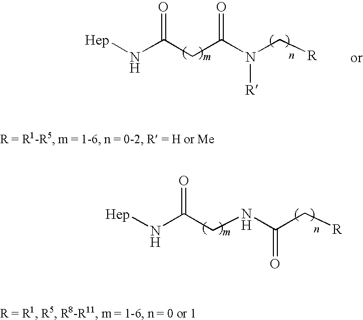

wherein R═R¹⁻¹¹ may be one of the following residues:
R¹=2,2,6,6-tetramethyl-1-oxylpiperidin-4-yl,
R²=3-amino-2,2,6,6-tetramethyl-1-oxylpiperidin-4-yl,
R³=4-alkyloxycarbonyl-2,2,6,6-tetramethyl-1-oxylpiperidin-4-yl,
R⁴=4-hydroxyimino-2,2,6,6-tetramethyl-1-oxylpiperidin-3-yl,
R⁵=2,2,5,5-tetramethyl-1-oxylpyrrolidin-3-yl,
R⁶=2,2,6,6-tetramethyl-1-oxylpiperidin-4-diyl,
R⁷=2,2,5,5-tetramethyl-1-oxylpyrrolidin-3-diyl, R⁸=2,2,6,6-tetramethyl-1-oxyl-1,2,5,6-tetrahydropyridin-4-yl,
R⁹=4-acetylamino-2,2,6,6-tetramethyl-1-oxylpiperidin-4-yl,
R¹⁹=2,2,5,5-tetramethyl-1-oxylpyrrolin-3-yl,
R¹¹=2,2,5,5-tetramethyl-4-bromo-1-oxylpyrrolin-3-yl,

### 3. Alternative Methods for Making Heparin-Nitroxide Derivatives

Another way for making the heparin-nitroxide derivatives of the invention relates to heparin —OH and/or —NH2 groups derivatisation under anhydrous conditions.

Nitroxyl radicals with carboxylic acid anhydride or acid chloride functional groups are suitable for —OH and/or —NH2 groups acylation of heparin salts with bulky ammonium cations in polar organic solvents (for details of analogous reactions see M. Petitou, C. Coudert, M. Level, J.-C. Lormeau, M. Zuber, C. Simenel, J.-P. Fournier, J. Choay, “Selectively O-acetylated glycosaminoglycan derivatives”, Carbohydr. Res., 1992, 236, 107-119). Isocyanato nitroxides can also be used for coupling of nitroxides to heparin through —OH and/or —NH2 groups under anhydrous conditions (W. Marconi, F. Benvenuti, A. Piozzi, “Covalent bonding of heparin to a vinyl copolymer for biomedical applications”, Biomaterials, 1997, 18, 885-890).

### Synthesis and Characterization of Heparin-Nitroxides

The progress of the reactions was monitored by HPLC, TLC, and EPR techniques. A Milikhrom chromatograph was used for HPLC (column 2×64 mm, Separon C18 (5 μm), detection at 240 nm) with the use of 30% aqueous MeCN containing KH2PO4 (0.05 M) as the eluent. TLC was carried out on Silufol UV254 0.25-mm silica gel plates. Visualization of the TLC plates was by one of the following methods: (1) UV light (254 nm), or (2) staining solution (0.3% ninhydrin in water containing 3% acetic acid) followed by heating. IR spectra were recorded in the range of 400 to 4000 cm⁻¹ on a Specord 75-IR spectrometer in Nujol. EPR spectra were measured at room temperature on an SE/X 2544 instrument at a UHF power of 2 mW and a modulation of 0.032 mT. Mass spectrum was recorded on a Finnigan-4021 (IE, 55 eV) apparatus.

For quantitative analysis, the samples of reaction mixtures were taken and the unbounded NRs were determined by HPLC and/or EPR after precipitation of the polymer by excess of MeCN. For heparin carboxyl group derivatisation, the fraction of modified heparin disaccharides was calculated as α=615 m/a, were 615 is the assumed average MW of disaccharide unit in unmodified heparin, m is the quantity (mM) of NR bonded to polymer (found as the difference between the quantities of the NR introduced into the reaction and the unbounded NR in the mother liquid after precipitation of the modified polymer at the end of the reaction) and a is the quantity of heparin taken for the reaction (mg).

For heparin N-desulfation and subsequent complete NH₂-group acylation, the fraction of modified heparin disaccharides was calculated as α=615 ml(a+102 m), were 102 is the difference in MW of unmodified and 100% N-desulfated heparin (—SO₃Na+H). The degree of heparin derivatisation was found also by double integration of the ESR spectra of the modified heparin (2-3 mg/ml water solutions) in comparison to the known concentration (5×10⁻⁴ M in water) of the reference radical 2,2,6,6-tetramethylpiperidine-1-oxyl. By comparison of the both methods of α value determinations, it was found that the integration method undervalue it by 0 to 20 relative %, presumably, due to the double integration error of the broadened spectra (the greater was the broadening, the higher was the deviation).

Heparin (H4784) was purchased from Sigma, trifluoroacetic anhydride, ethyl chloroformate, N-hydroxy succinimide and N-(3-dimethylaminopropyl) N′-ethylcarbodiimide hydrochloride were purchased from Aldrich and utilized as received. Nitroxyl radicals 4-amino-2,2,6,6-tetramethylpiperidine-1-oxyl, 3-amino-2,2,5,5-tetramethylpyrrolidine-1-oxyl, 4-oxo-2,2,6,6-tetramethylpiperidine-1-oxyl, 3-oxo-2,2,5,5-tetramethylpyrrolidine-1-oxyl and 2,2,6,6-tetramethylpiperidine-1-oxyl were synthesized according to the described methods (Rozantsev E G, “Free nitroxyl radicals”, Plenum Pres, New York, 1970), 2,2,6,6-tetramethyl-4-(succinimidooxycarbonylmethyl)piperidine-1-oxyl was made according to (Maksimova L A, Grigoryan G L, Rozantsev E G, Rus. Chem. Bull., 1975, 24: 859-862) and 4-hydrazono-2,2,6,6-tetramethylpiperidine-1-oxyl according to (H. Schlude, “A new reagent for the spin labeling of aldehydes and ketones”, Tetrahedron Lett., 1976, 25, 2179-2182). Solvents were purified by standard procedures and distilled.

The structure of the heparin-nitroxide derivatives of the invention is in agreement with their EPR- and IR-spectra (see Figures). Antioxidant nitroxide-heparin agents with the general formula Hep-L-NR are characterized by three-line EPR-spectra, which arise from the splitting of an unpaired electron signal on the 14N-nucleus.

The degree of broadening of the lines in the EPR-spectra depends on the nature of the linker L. Nitroxyl radicals coupled to the macromolecule with long flexible linkers have greater mobility and exhibit more narrow lines spectra. Compared to unmodified heparin, the IR-spectra of nitroxide heparin agents exhibit a new band at ˜1550 cm⁻¹ (—CO—NH—, amide II band), which is indicative of amide bond formation according to the reaction scheme above. The amide I band due to the C═O stretching vibrations overlaps with the broad band of the heparin —CO₂-groups (1625 cm⁻¹ for unmodified heparin) and the maximum of the resulting band is at 1640-1655 cm⁻¹ (see Examples).

Biological and Paramagnetic Properties

As such the antioxidant nitroxide-heparin agents of the invention are excellent tools for both therapeutic applications and EPR/MR imaging. The antioxidant nitroxide heparin agents of the invention describe for the first time an extracellular-superoxide-dismutase mimetic.

The invention also encompasses a pharmaceutical composition, comprising an heparin-nitroxide derivative of the invention. The pharmaceutical composition may contain any suitable carrier, solvent, vehicle or excipient for stable storage and efficient delivery of the therapeutic agent to its target.

The heparin-nitroxide derivative of the invention can be used as therapeutic agent for the preparation of a medicament for treatment of acute and chronic diseases that are associated with oxidative extracellular stress. Accordingly, the invention also concerns a pharmaceutical composition, comprising a heparin-nitroxide derivative of the invention, and a pharmaceutically acceptable carrier. Any suitable carrier can be used that is known in the art.

In particular, the disease associated with extracellular oxidative stress to be treated by the heparin-nitroxide derivative of the invention is selected from the group consisting of oxidative stress-dependent platelet activation, cardiovascular disease, neurodegenerative diseases such as Alzheimer's, pulmonary disease, thrombosis, chronic inflammatory disease, diabetes, ischemia, rheumatoid arthritis, cardiac infarct, cancer, hypertension, ocular damage, ischemia-reperfusion injury, and septic shock. In addition, the heparin-nitroxide derivative of the invention may also be used for the preservation of biological transplants.

A further aspect of the invention concerns the use of the heparin-nitroxide derivative of the invention as a contrast agent for MRI and as an EPR active redox-, pH-, or oxymetry-probes in EPRI. Potentially, the heparin-nitroxide derivative of the invention is suitable monitoring local vascular oxidative stress by means of EPRI approach.

Accordingly, the invention comprises in a further aspect a method for electron paramagnetic resonance imaging (EPRI) of vascular structure in biological vessels, in particular the vascular intima of conductive blood vessels, comprising:

(a) contacting vascular tissue with a paramagnetic heparin-nitroxide derivative of the invention,
(b) washing of unbound heparin-nitroxide derivative,
(c) measuring of EPR signals obtained from vascular-bound paramagnetic heparin-nitroxide derivative.

The inventors found that the heparin-nitroxide in solution exhibits an EPR signal that is typical for nitroxyl radicals. The signal is slightly broader than the EPR signal of TEMPOL. In addition, heparin-nitroxide binds to vascular tissue and cannot be washed out by Krebs solution. However, it can be replaced by conventional heparin, indicating the competition for the same binding site.

For example, rat aorta contains about 10⁶-10⁷ binding sites per cell that can be efficiently occupied by the heparin-nitroxide derivatives of the invention. As further shown by the inventors, the antioxidant activity of the heparin-nitroxide derivate of the invention is comparable to the cyclic nitroxide TEMPOL. At low concentrations, heparin-nitroxide effectively prevents the binding of myeloperoxidase (the most devastating free radical generating enzyme) to human umbilical vein endothelial cells (HUVECs).

The antioxidant and paramagnetic properties of the heparin-nitroxide derivative (in short: H—NR) of the invention will be more apparent in the light of the following experiments, the results of which are shown in the accompanying Figures.

### BEST MODE FOR CARRYING OUT THE INVENTION

### Examples

### Example 1

Synthesis of 4-[(5-aminopentyl)carbonylamino]-2,2,6,6-tetramethylpiperidine-1-oxyl. Mixture of 1.31 g (10 mM) of 6-aminocaproic acid and 4.5 ml of trifluoroacetic anhydride was heated for 2 h at 80° C. in soldered ampoule. Volatile components of the reaction mixture were evaporated at reduced pressure. The residual liquid consisted mainly of bis-trifluoroacetylated 6-aminocaproic acid. To achieve hydrolysis of mixed anhydride function of this intermediate, 0.25 ml (14 mM) of water was added to it with ice cooling. The solution was left for 1 h at ˜20° C. and after that it was azeotroped with three 10 ml portions of dry benzene. The yield of 6-(trifluoroacetylamino)caproic acid was 2.27 g, mp 83° C. It was dissolved in 10 ml of ethyl acetate and triethylamine (1.39 ml, 10 mM) and ethyl chloroformate (0.96 ml, 10 mM) were added sequentially at ice bath cooling and stirring. After stirring for 20 min at the same cooling, solution of 4-amino-2,2,6,6-tetramethylpiperidine-1-oxyl (1.71 g, 10 mM) in 2.5 ml of ethyl acetate was added in 5 min time interval. The mixture was allowed to warm to room temperature and stirred for additional 30 min. The triethylammonium chloride salt was filtered off and washed with ethyl acetate (3 ml×3). Red ethyl acetate solution was washed sequentially with 2 ml of 0.1 M HCl, 1 ml of water and 2 ml of saturated NaHCO₃ solution and dried over anhydrous MgSO₄. Ethyl acetate was removed in vacuo to yield 3.9 g of 4-[(5-trifluoroacetylaminopentyl)carbonylamino]-2,2,6,6-tetramethylpiperidine-1-oxyl as a red oil. It was dissolved in 7 ml of ethanol and 11 ml of 1 M NaOH solution in water was added at ˜10° C. The mixture was left at room temperature for 20 h. Solid K2CO3 (10 g) and 10 ml of ethyl acetate were added with stirring. The upper organic layer was separated, washed with saturated aqueous solution of NaCl, dried over anhydrous MgSO₄ and the solvent was removed in vacuo. The product was purified by column chromatography (silica gel, 5:1 to 2:1 chloroform/methanol) yielding 2.3 g of 4-[(5-aminopentyl)carbonylamino]-2,2,6,6-tetramethylpiperidine-1-oxyl as red hygroscopic solid, which did not have an sharp melting point. Found (%): C, 62.95; H, 10.55; N, 14.35. C₁₅H₃₀N₃O₂. Calculated (%): C, 63.34; H, 10.63; N, 14.77. MW 284.421; IR (Nujol mull), v/cm−1: 1543 (O═CNH), 1645 (C═), 3265 (OCN—H), 3335, 3445 (NH2). MS (55 eV), m/z (I, %): 285 [M+1]+(4.5), 284 [M]+(0.6), 198 (24), 155 (2.5), 140 (16), 124 (13), 114 (11), 109 (11), 98 (11), 84 (100), 70 (38), 55 (43), 44 (49), 43 (65). EPR(H2O, 5·10−4 M): three lines, g factor was 2.0056, aN=1.70 mT.

### Example 2

Coupling of amino nitroxide to the heparin carboxyl groups. 171 mg (1 mM) of 4-amino-2,2,6,6-tetramethylpiperidine-1-oxyl (H2N—R1) and 115 mg of N-hydroxy succinimide (NHS) were mixed with solution of 615 mg of heparin sodium salt in 10 ml of 0.1 M HCl. The solution obtained was cooled in an ice bath and N-(3-dimethylaminopropyl) N′-ethylcarbodiimide hydrochloride (EDC) (230 mg, 1.2 mM) was added with stirring. The resulting solution was stirred in the ice bath for 30 min and after that stirring was continued at ˜20° C. H₂N—R1 consuming was monitored by HPLC. Optimal pH˜5 was maintained by adding of 0.1 M NaOH solution. After 7 h 120 mg (0.70 mM) of H₂N—R1 was coupled to heparin. The reaction mixture was freeze dried until it weight was ˜4 g and absolute ethanol (30 ml) was added slowly with stirring. The precipitate was triturated to powder, filtered, washed with absolute ethanol (3 ml×3), dissolved in water (3 ml) and reprecipitated with absolute ethanol (30 ml). The precipitate was washed with absolute ethanol and vacuum dried, yielding 660 mg of Hep-C(O)NH—R1 as a pale pink powder. The nitroxide radical content in the product was found to be 1.14×10⁻³ M/g (mol of R1 per gram of the derivate). For disaccharide unit assumed MW=615, it meant 70% of heparin carboxyl groups derivatisation. IR of Hep-C(O)NH—R1 (Nujol mull), v/cm−1: 1000, 1030, 1235 (SO3-), 1550(O═CNH), 1655 (O═CNH+CO2-). EPR(H₂O, 3 mg/ml): three lines with 100:115:60 heights ratio, g factor was 2.0056, aN=1.70 mT.

### Example 3

Using the method of Example 2,3-amino-2,2,5,5-tetramethylpyrrolidine-1-oxyl (H2N—R5) was coupled to heparin, time of reaction was 4.5 h. The nitroxide radical content in the product was found to be 1.31×10⁻³ M/g or 81% of heparin carboxyl groups derivatisation. IR of Hep-C(O)NH—R5 (Nujol mull), v/cm−1: 1000, 1030, 1235 (SO3-), 1553(O═CNH), 1657 (O═CNH+CO2-). EPR(H2O, 3 mg/ml): three lines with 100:130:50 heights ratio, g factor was 2.0053, aN=1.58 mT.

### Example 4

Using the method of Example 2, 4-[(5-aminopentyl)carbonylamino]-2,2,6,6-tetramethylpiperidine-1-oxyl[H₂N(CH2)5C(O)NH—R1] was coupled to heparin, time of reaction was 24 h. The nitroxide radical content in the product was found to be 7.5×10−4 M/g. For disaccharide unit estimated MW=615, it meant 46% of heparin carboxyl groups derivatisation. IR of Hep-C(O)HN(CH2)5C(O)NH—R1 (Nujol mull), v/cm−1: 1000, 1030, 1235 (SO3-), 1548(O═CNH), 1645 (O═CNH+CO2-). EPR (H2O, 2.7 mg/ml): three lines with 100:101:68 heights ratio, g factor was 2.0056, aN=1.70 mT.

### Example 5

Using the method of Example 2,4-hydrazono-2,2,6,6-tetramethylpiperidine-1-oxyl (H2NN═R6) was coupled to heparin, time of reaction was 5 h. The nitroxide radical content in the product was found to be 1.18×10⁻³ M/g. For disaccharide unit estimated MW=615, it meant 73% of heparin carboxyl groups derivatisation. IR of Hep-C(O)NHN═R6 (Nujol mull), v/cm−1: 1000, 1030, 1235 (SO3-), 1570(0=CNH), 1655 (O═CNH+CO2-). EPR(H2O, 5 mg/ml): three lines with 100:140:60 heights ratio, g factor was 2.0056, aN=1.70 mT.

### Example 6

Heparin sodium salt (62 mg) was dissolved in 1 ml of 0.1 M hydrazine monohydrochloride in water. To this solution NHS (16 mg, 0.15 mM) and EDC (20 mg, 0.1 mM) were added sequentially and the mixture was left for 20 h at ˜20° C. The reaction mixture was freeze dried until it weight was ˜0.25 g and absolute ethanol (4 ml) was added slowly with stirring. The precipitate was triturated to powder, filtered, washed with absolute ethanol (2 ml×2), and vacuum dried, yielding 66 mg of Hep-C(O)NHNH2. It was dissolved in 0.5 ml of water, 4-oxo-2,2,6,6-tetramethylpiperidine-1-oxyl (0=R6) (43 mg, 0.25 mM) was added to the solution and the mixture was kept at ˜20° C. 0=R6 consuming was monitored by HPLC. After 20 h 14 mg (0.082 mM) of H2N—R1 was coupled to heparin. The reaction mixture was freeze dried until it weight was ˜0.2 g and absolute ethanol (4 ml) was added slowly with stirring. The precipitate obtained was washed with absolute ethanol (1 ml×2) and vacuum dried, yielding 53 mg of Hep- Hep-C(O)NHN═R₆ as a pale yellow powder. The nitroxide radical content in the product was found to be 1.34×10⁻³ M/g and meant 70% of heparin carboxyl groups derivatisation. The IR and EPR characteristics of the derivate were the same as in example 5.

### Example 7

Using the method of Example 6,3-oxo-2,2,5,5-tetramethylpyrrolidine-1-oxyl (O═R7) was coupled to heparin, time of reaction was 20 h. The nitroxide radical content in the product was found to be 1.46×10⁻³ M/g and meant 90% of heparin carboxyl groups derivatisation. IR of Hep-C(O)NHN═R7 (Nujol mull), v/cm−1: 1000, 1030, 1235 (SO3-), 1575(0=CNH), 1655 (O═CNH+CO2-). EPR(H2O, 5 mg/ml): three lines with 100:137:62 heights ratio, g factor was 2.0053, aN=1.57 mT.

### Example 8

N-desulfation of heparin was performed by modification of described methods (D. S. Milbrath, R. H. Ferber, W. E. Barnett, “Diagnostic radio-labeled polysaccharide derivatives”, U.S. Pat. No. 4,385,046, 5/1983; A. I. Usov, K. S. Adamyants, L. I. Miroshnikova, A. A. Shaposhnikova, N. K. Kochetkov, “Solvolytic desulphation of sulphated carbohydrates”, Carbohydr. Res., 1971, 18, 336-338; Y. Inoue, K. Nagasawa, “Selective N-desulfation of heparin with dimethyl sulfoxide containing water or methanol”, Carbohydr. Res., 1976, 46, 87-95; L. Huang, R. J. Kerns, “Diversity-oriented chemical modification of heparin: Identification of charge-reduced N-acyl heparin derivatives having increased selectivity for heparin-binding proteins”, Bioorg. Med. Chem., 2006, 14, 2300-2313). Sodium heparin (280 mg) was converted to the pyridinium salt by dissolving in water and passing through cationic exchange resin (Dowex 50WX4-400, H+ form) at the ice bath cooling. Double excess of pyridine was added relative to the total number of the acidic groups in heparin (the final pH ˜5). The solution obtained was frozen and lyophilized. The pyridinium heparin obtained (288 mg) was dissolved in 10 ml DMSO/water (95:5) and stirred in 35° C. water bath for 70 min. Ice-cooled water (10 ml) was added and the pH was adjusted to 9 with 0.2 N NaOH at ice-cooling. The solution was dialyzed against water (MWCO=2000) and rotary evaporated at 20° C. water bath to residual weight of ˜0.7 g. Absolute alcohol (5 ml) was added with mixing. The precipitate was filtered, washed with absolute ethanol (3 ml×2) and vacuum dried, yielding 203 mg of partially N-desulfated heparin Hep-NH₂. It was dissolved in 10 ml of DMSO/H₂O (2:1), saturated with NaHCO₃. 94 mg (0.30 mM) of 2,2,6,6-tetramethyl-4-(succinimidooxycarbonylmethyl)piperidine-1-oxyl (crystallized from ethanol, mp 172-174° C.) was pounded with a pestle and added to Hep-NH2 solution with stirring (˜1 equiv of NHS ester per 1 equiv disaccharide unit). Mixing was continued at ˜20° C.

By HPLC and EPR analysis of the reaction mixture after 12 and 24 h it was found that 1) 0.23 (12 h) and 0.24 (24 h) mM of NHS ester was coupled to heparin; 2) some quantity of the starting NHS ester still existed in the mixture after 24 h reaction time; 3) pH of the reaction mixture was in the range 8.5-9. From this data it was concluded that all free amino groups of Hep-NH2 were acylated.

The volume of reaction mixture was halved by freeze drying and the polymer was precipitated by addition of excess of acetone/ether (1:1), centrifuged, decanted and washed with acetone. Dried polymer was dissolved in water (1.5 ml), centrifuged, decanted and re-precipitated with absolute ethanol (15 ml). The precipitate was washed with absolute ethanol and vacuum dried, yielding 189 mg of Hep-NHC(O)CH2—R1 as a pale orange powder. The fraction of modified disaccharides in the product a was found to be 0.65. It meant 65% disaccharide modification and the nitroxide radical content in the product equal to 9.6·10−4 M/g. IR of Hep-NHC(O)CH2-R1 (Nujol mull), v/cm−1: 985, 1020, 1240 (SO3-), 1535(NHC═O), 1640 (NHC═O+CO2-). EPR(H2O, 2.1 mg/ml): three lines with 100:127:20 heights ratio, g factor was 2.0055, aN=1.71 mT.

### Example 9

### Comparison Example: Specificity of Dextran-Nitroxide and Heparin-Nitroxide

To demonstrate that dextran cannot bind to the specific heparin-binding sites in vascular tissue, the following experiment has been performed: Rat aorta was pretreated with heparin-nitroxide in the absence or in the presence of molar excess of dextran (M.W. 20000). As shown in the FIG. 12, even 100 molar excess of dextran was unable to replace heparin-nitroxide from the vascular tissue. Therefore, the heparin-nitroxide derivative of the present invention is able to specifically bind with high affinity to specific sites in biological vessels.

### DESCRIPTION OF THE FIGURES

FIG. 1. General structure of the antioxidant heparin-nitroxides agents of the invention. There are two principal ways for conjugation of heparin with cyclic nitroxides that are preferred in the present invention: derivatisation of heparin via carboxyl groups (A) and derivatisation of heparin via amino groups (after N-desulfation) (B).

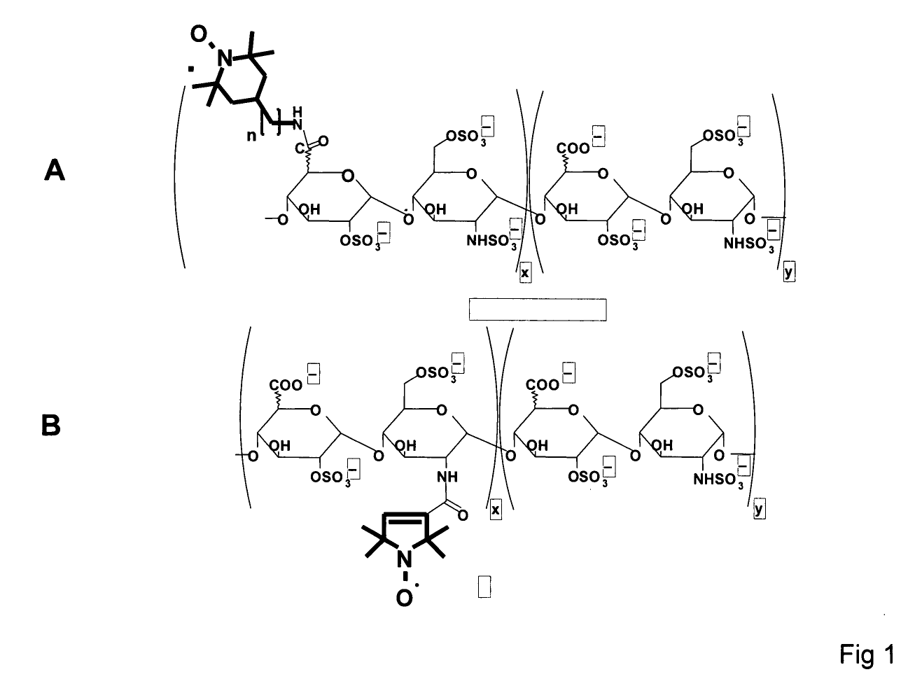

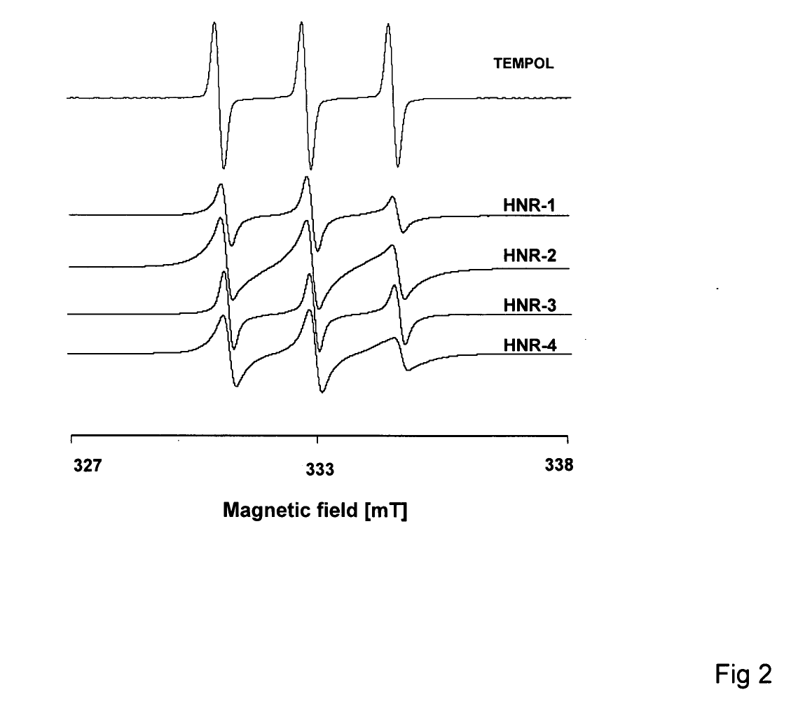

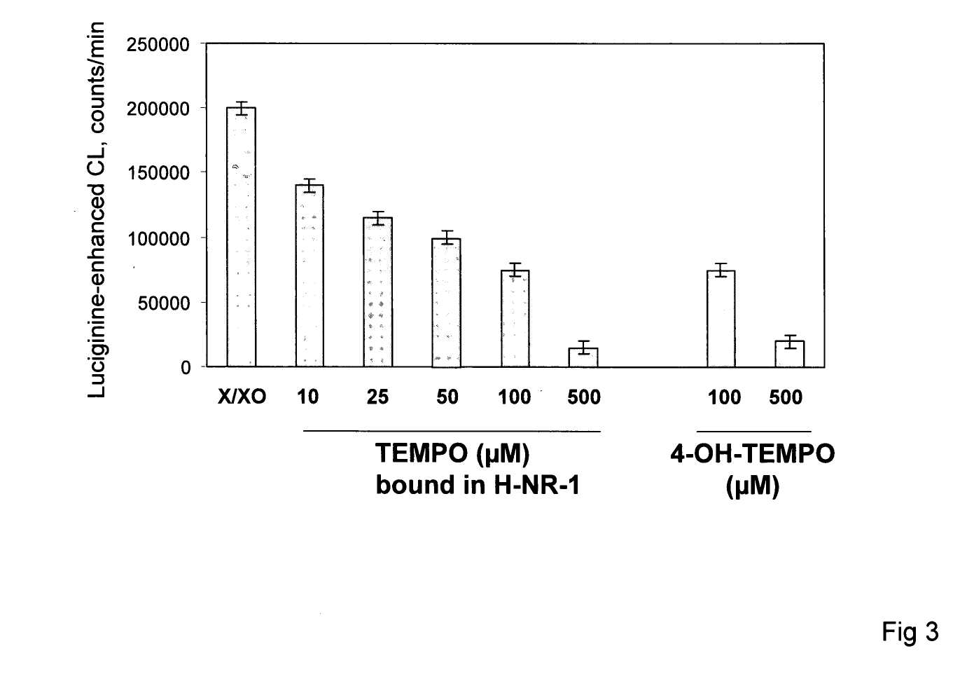

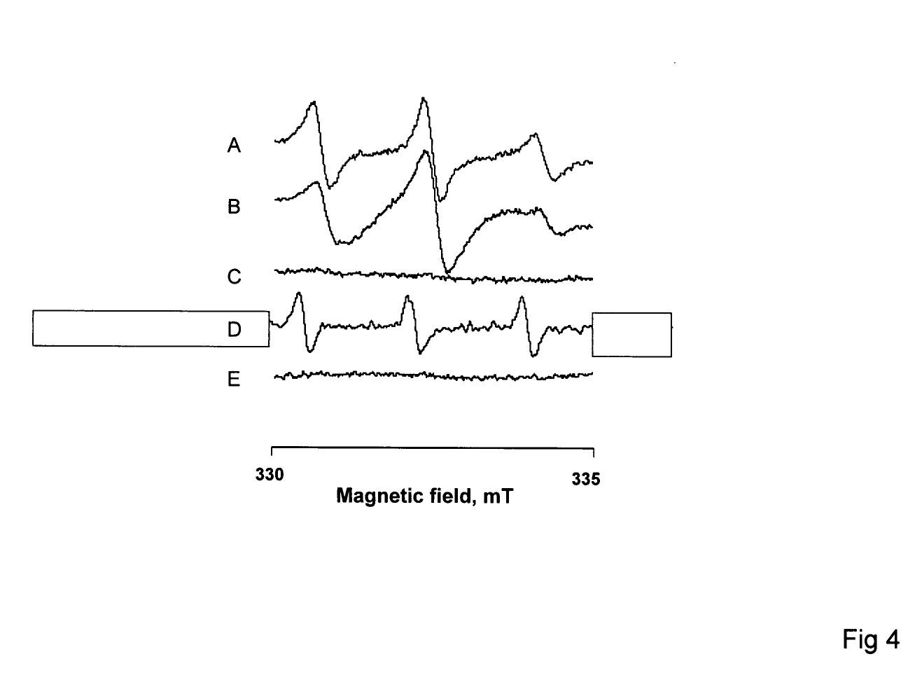

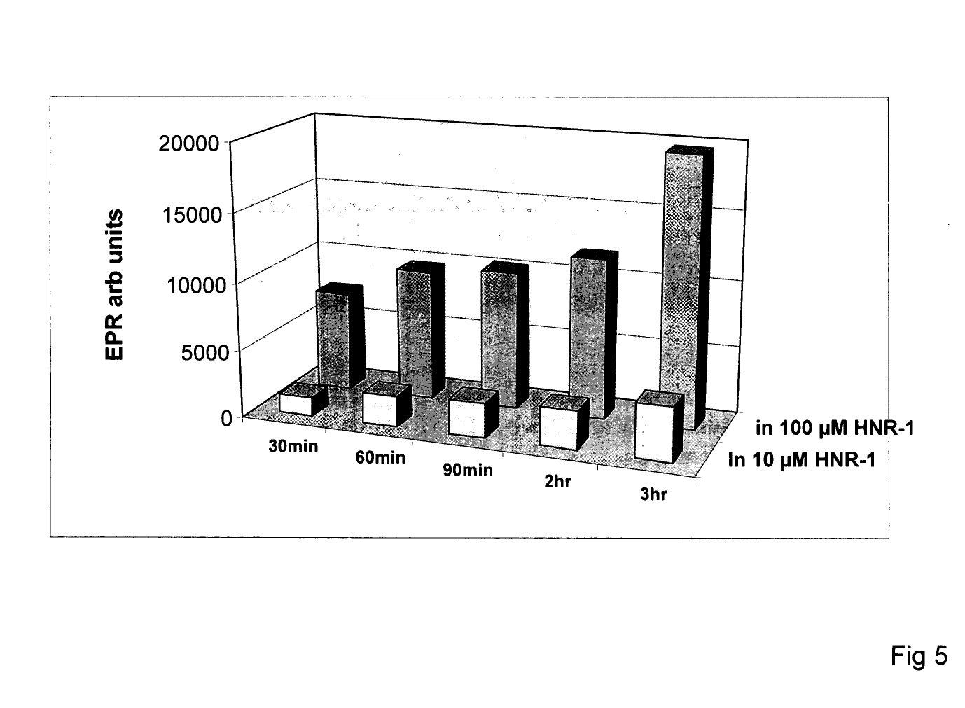

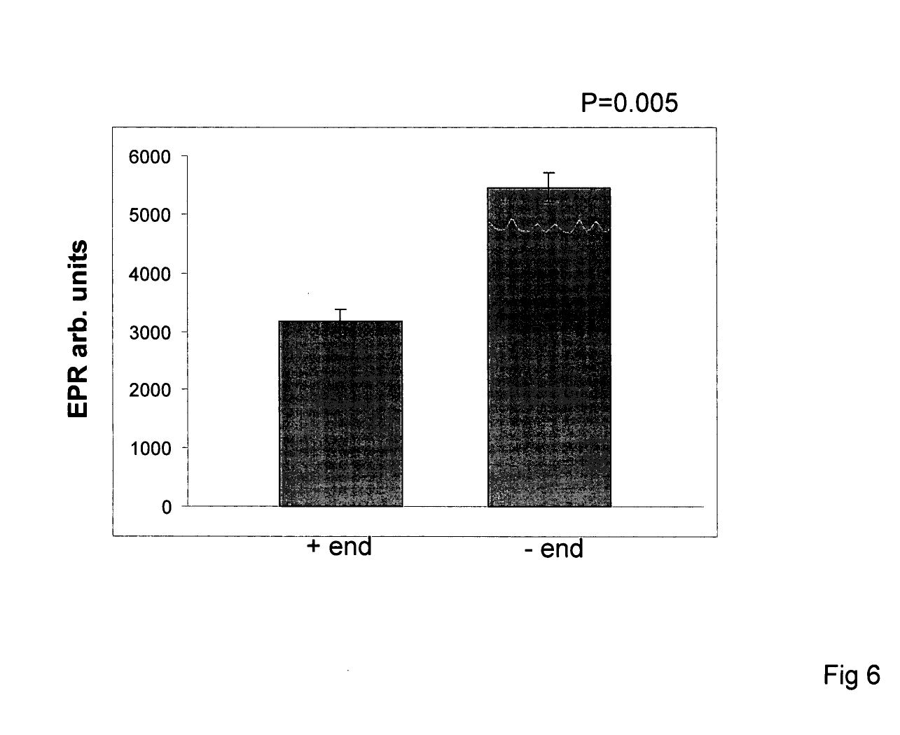

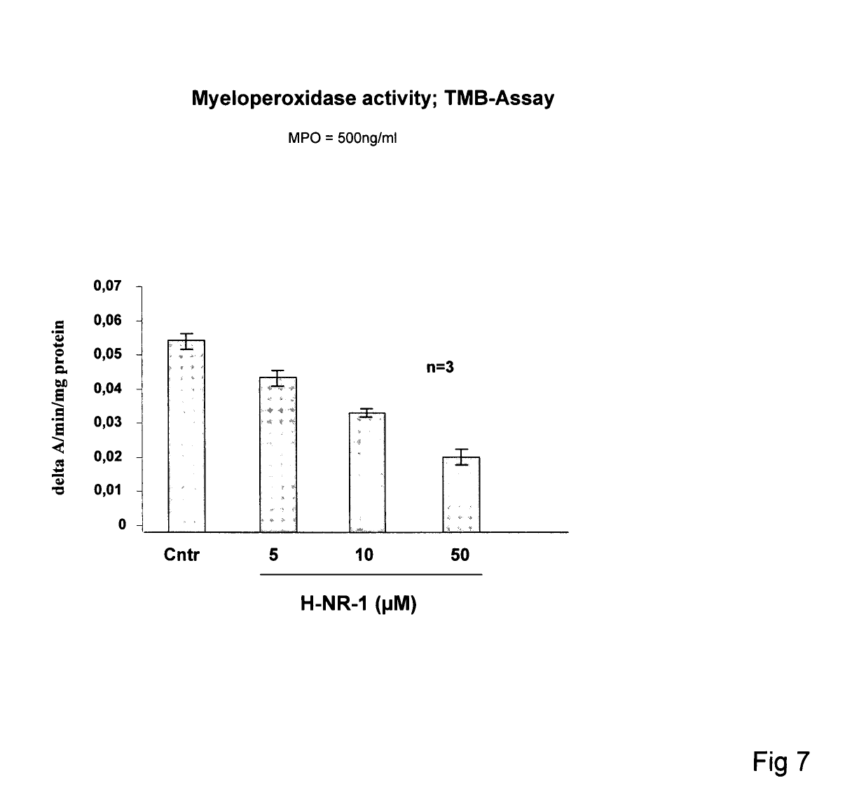

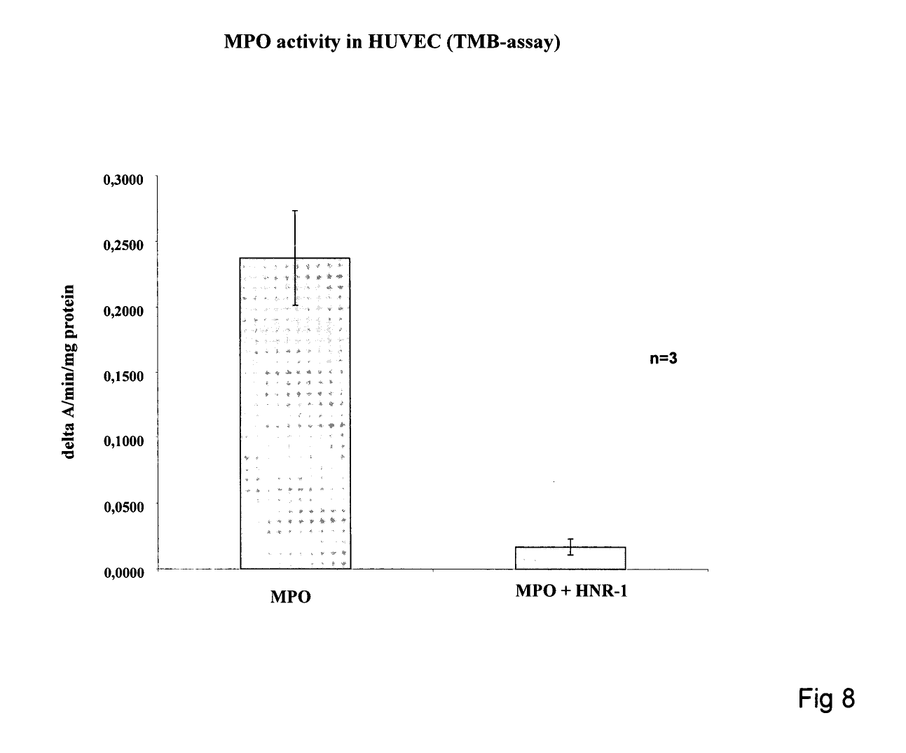

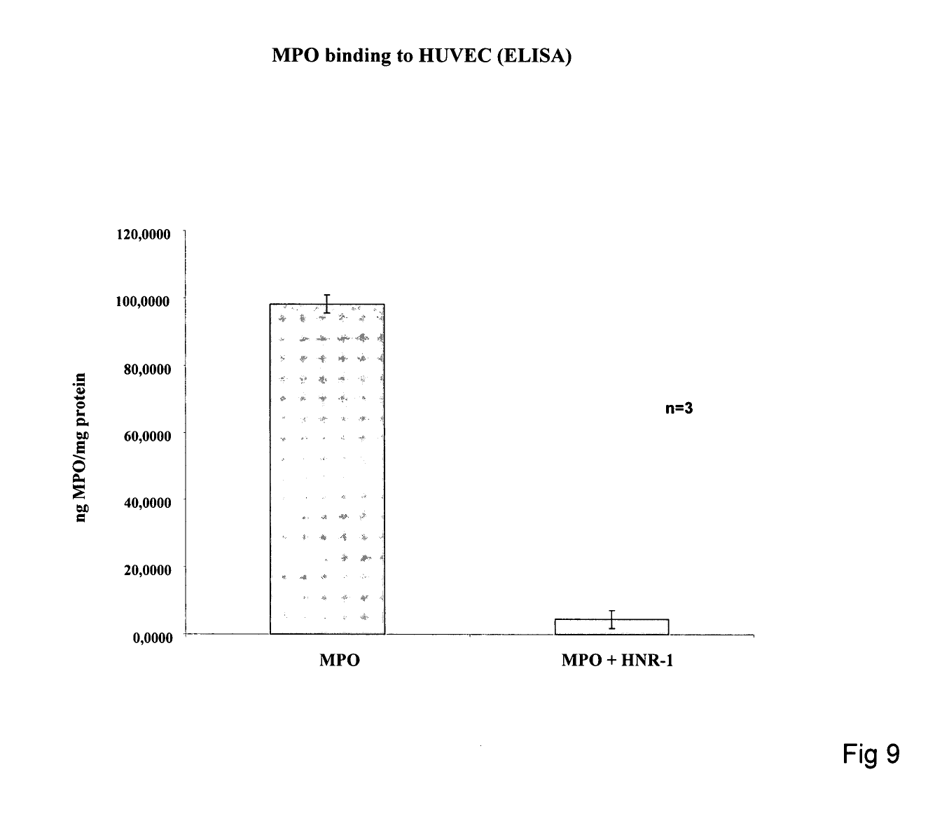

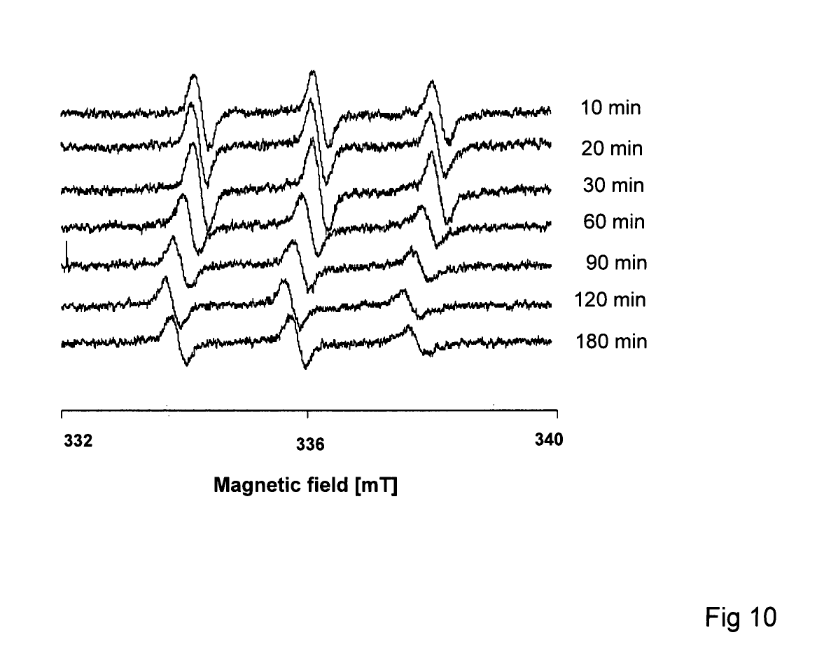

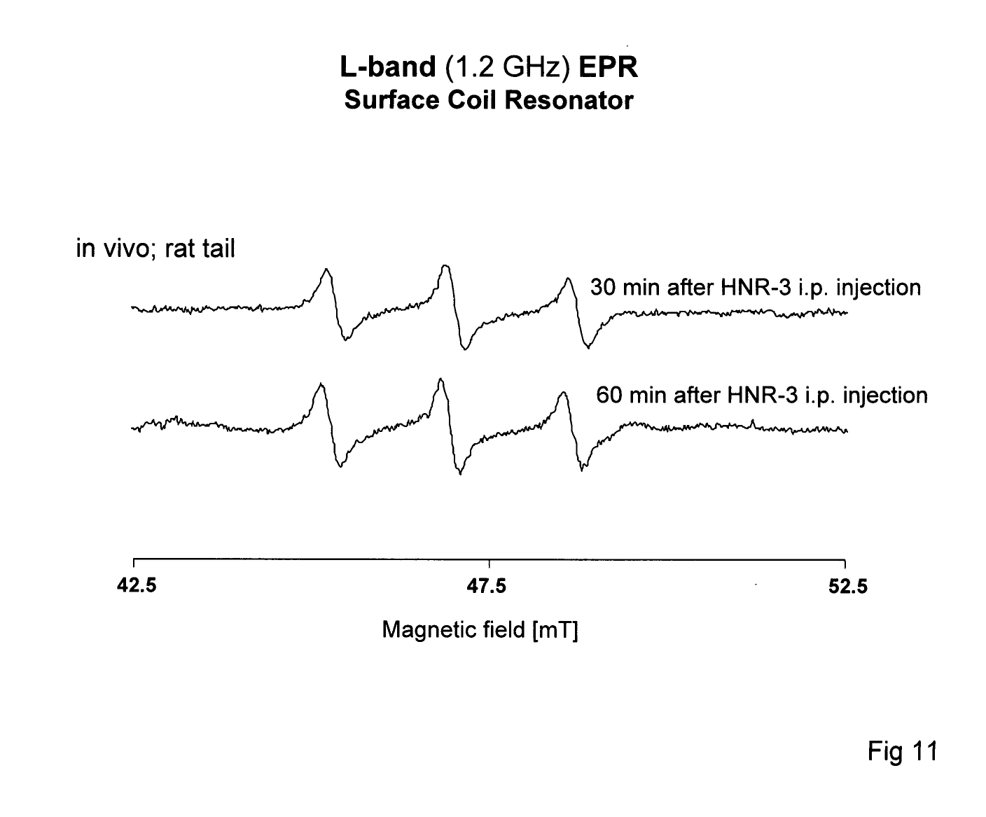

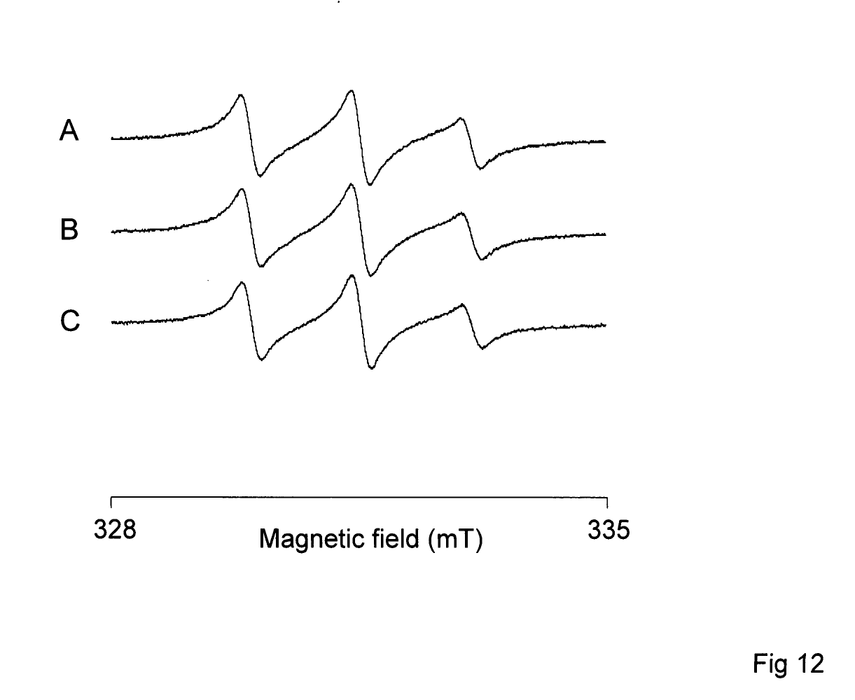

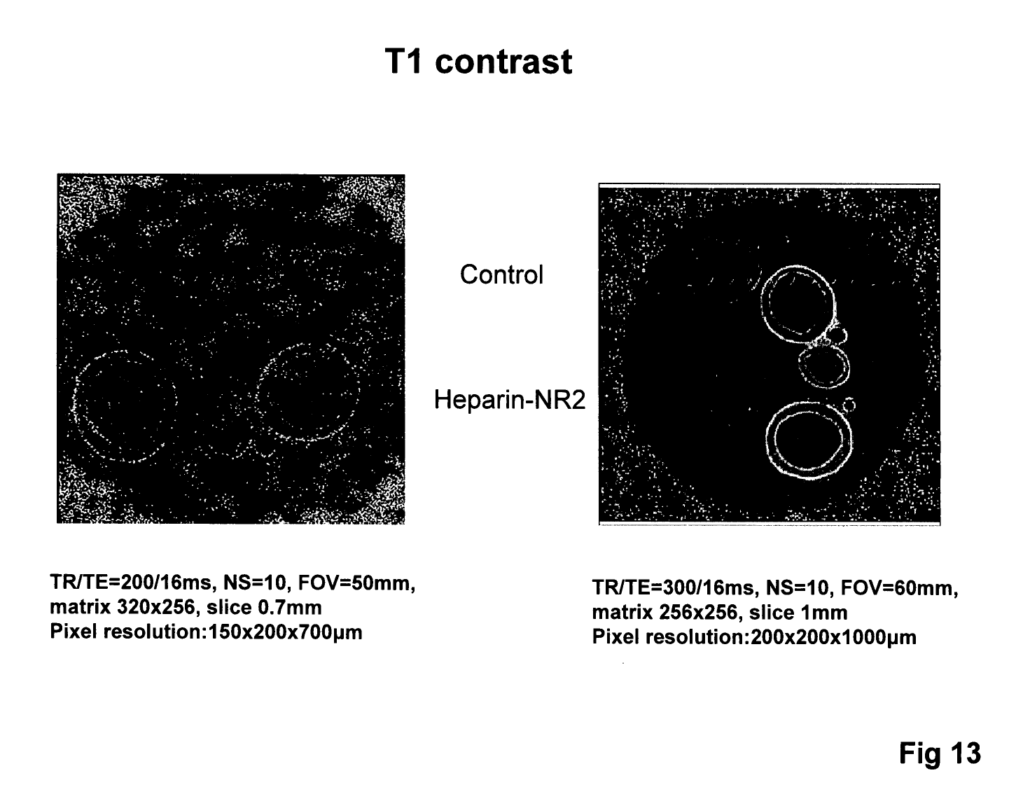

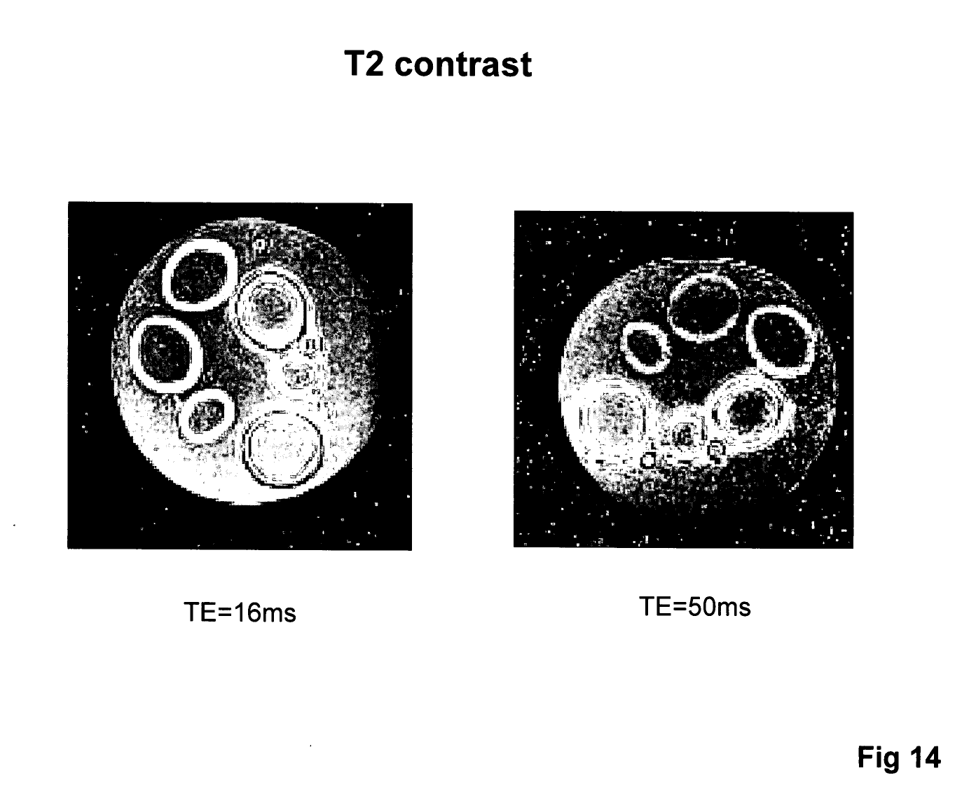

The biological properties and EPR characteristics were studied using the following antioxidant heparin-nitroxide (H—NR) preparations:

H—NR-1: Nitroxide conjugated with heparin via carboxyl group without long linker (20% disaccharides modified by TEMPO, FW˜18000).

H—NR-2: Nitroxide conjugated with heparin via carboxyl group without long linker (72% disaccharides modified by TEMPO, FW˜18000).

H—NR-3: Nitroxide conjugated with heparin via carboxyl group with a long linker (Hep-C(O)HN(CH₂)₅C(O)NH—R¹) (45% disaccharides modified by TEMPO, FW˜18000).

H—NR-4: Heparin conjugation with nitroxide via amino group without long linker (65% disaccharides modified by TEMPO, FW ˜18000).

H and Hep, respectively, designate heparin. In Hep-C(O)NH—R¹ the group C(O)NH functions as a linker. FW designates the molecular weight of the whole construct, i.e. H—NR. Preferably, heparin is used with a molecular weight of around 15 KDa.

FIG. 2. Spectra EPR of TEMPO and different H—NRs (0.1 mM aqueous solutions, pH 7.4). EPR spectra were recorded at room temperature using an X-band radiospectrometer MS200 (Magnettech GmbH, Berlin). Instrument parameters were 10 mW microwave power, 0.1 mT amplitude modulation, 100 kHz modulation frequency, sweep field 11 mT and 120 s sweep time.

In solution all four H—NRs exhibited triplet EPR signals (a_{N}˜17 gauss), which is typical for nitroxyl radicals. The line widths of the EPR signals of all H—NRs were slightly broader in comparison to the EPR signal of TEMPOL, indicating slight decrease in the mobility of nitroxyl groups upon binding to heparin as well as spin-spin interactions between them. Thus, ΔH (in gauss) were 1.8, 2.1, 2.5, 2.0 and 2.5 for TEMPOL, H—NR-1, H—NR-2, H—NR-3 and H—NR-4, respectively. The line width the signal is critical for the in vivo EPR imaging. A narrower signal provides for better resolution, and is therefore preferred. As a result, the H—NR-3, in which nitroxide is bound to heparin via a linker, provides for a higher spectral mobility, and is thus preferred for EPR imaging.

FIG. 3. Comparison of the superoxide scavenging properties of the heparin bound TEMPO groups and 4-hydroxy-TEMPO. Superoxide production was generated in the xantine (X, 0.5 mM) plus xantine oxidase (XO, 50 mU/ml) system (pH 7.4). The chemiluminescence signals were recorded in the presence of 50 μM lucigenin, 1 mM DTPA and a test compound using chemiluminometer Lumat 9507. The concentration of the heparin-bound TEMPO groups was calculated from the known content of the TEMPO-modified disaccharides (20% in H—NR-1). Mean±SEM are shown for 4 different measurements.

TEMPO is known to be an excellent antioxidant acting via scavenging of superoxide in a similar manner as superoxide dismutase. Thus, the inventors compared the superoxide scavenging activity of H—NR and TEMPO using the lucigenin-enchanced chemiluminescence assay and the superoxide generation system: xantine plus xantine oxidase.

As shown in FIG. 3, the superoxide scavenging activity of TEMPO groups-bound to heparin was comparable to the activity of 4-Hydroxy-TEMPO. Thus, in homogenous aqueous solution, the binding of TEMPO groups to heparin does not change its reactivity to superoxide. For the first time the inventors received a heparin agent with antioxidant and paramagnetic properties.

FIG. 4. Spectra EPR, (A) 10 μM H—NR-1 solution; (B) rat aortic ring (3 mm long) pre-incubated with 10 μM H—NR-1 for 1 hour and then washed 3 times with Krebs buffer; (C) rat aortic ring (3 mm long) pre-incubated with 10 μM H—NR-1 in the presence of heparin (liquemin, 30 units/ml) for 1 hour and then washed 3 times with Krebs buffer; (D) 5 μM 4-hydroxy-TEMPO; (E) rat aortic ring (3 mm long) pre-incubated with 10 μM 4-hydroxy-TEMPO for 1 hour and then washed 3 times with Krebs buffer. EPR spectra were recorded at room temperature using an X-band radiospectrometer MS200 (Magnettech GmbH, Berlin). Instrument parameters were 10 mW microwave power, 0.1 mT amplitude modulation, 100 kHz modulation frequency, sweep field 5 mT and 60 s sweep time. Representative spectra of 3 experiments are shown.

The modification of heparin by nitroxide (especially via carboxyl group) may change the biological properties of heparin, i.e. its affinity to the heparan sulphate proteoglycans of the vascular extracellular matrix. The experiments have unequivocally shown that H—NR-1 sticks to rat aorta with high affinity and cannot be washed out thereafter with Krebs solution (FIG. 4B). The fact that the H—NR-1 EPR signal was absent in aorta but in the presence of excess of heparin (FIG. 4 C) indicates that H—NR-1 and non-modified heparin compete for the same binding sites. In contrast, incubation of aortas with 4-hydroxy-TEMPO did not result in the appearance of the EPR signal in tissue (FIG. 4D).

According to the inventors' calculations, rat aorta contains about 10⁶-10⁷ binding sites per cell. The half-life of the heparin-nitroxide in isolated vascular tissue is several hours at 37° C. These results demonstrate that the novel, EPR visible, heparin with antioxidant properties, can be efficiently targeted to vascular tissue and remains bound at extracellular sites.

FIG. 5. The binding of H—NR-1 to rat aorta as a function of time and concentration. Rat aortic rings (3 mm long) were incubated (37° C.) with 10 μM or 100 μM H—NR-1 either for 0.5 hr, 1 hr, 1.5 hr, 2 hr, 3 hr and then washed out with Krebs solution. To estimate the amount of H—NR-1 bound to tissue, aortic rings were placed in capillaries, and spectra EPR(X-band) were recorded.

The results demonstrate the concentration and time dependent accumulation of H—NR in rat aorta, and reflect the rate of penetration of H—NR into the vascular wall.

FIG. 6. Enhanced binding of H—NR in the endothelium-denuded rat aorta. Rat aortic rings (3 mm long) with or without endothelium were incubated (37° C.) with 10 μM H—NR-1 for 1 hr and then washed out with Krebs solution. To estimate the amount of H—NR-1 bound to tissue, aortic rings were placed in capillaries and spectra EPR(X-band) were recorded at room temperature.

The results obtained in these experiments suggest that the endothelium acts as a barrier for H—NR-1 penetration into the vascular wall. The large macromolecule heparin is known to have affinity not only to the endothelial cell surface but also to a variety of vascular extracellular matrix proteins including fibronectin and collagen. Vascular pathology is known to be associated with the endothelium insufficiency and remodelling of extracellular matrix. Potentially H—NR-1 can be preferably accumulated in the problematical sites of the vascular wall (i.e. early stages of atherosclerotic plaques) and by this way provides the local antioxidant defense in strategically important site. On the other hand, the EPR detection of the disturbed signal parameters/kinetic in a problematic zone, potentially can be used for a diagnostic purpose.

FIG. 7. Myeloperoxidase (MPO, Sigma; 500 ng/ml) activity was measured in the presence of increasing concentrations of H—NR-1 using the 3,3′,5,5′-tetramethylbenzidine (TMB) assay kit (Calbiochem). The absorbance of the oxidized TMB was detected at 450 nm. Measurements were performed in the laboratory of Dr. S. Baldus (University Hospital Hamburg-Eppendorf).

MPO has emerged as a critical mediator of inflammatory vascular diseases, such as atherosclerosis. MPO has been show to be accumulated in atherosclerotic plaques where it can oxidise high-density lipoproteins, activate metalloproteinases and exert cytokine-like property. These results indicate that H—NR can directly inhibit the MPO activity (presumably via scavenging of MPO derived oxidants) and thus H—NR may become an effective drug against the myeloperoxidase-dependent vascular tissue damage.

FIG. 8. Effect of H—NR-1 on the myeloperoxidase (MPO) activity (TMB assay) in human umbilical vein endothelial cells (HUVEC) lysate. HUVEC were pre-incubated with myeloperoxidase (MPO; 1 μg/ml) for 1 hr; then non-bound MPO was washed out and cells were further incubated in the presence or absence of H—NR-1 (50 μM) for additional 30 min. After the incubation cells were washed out, lysed and MPO activity was measured using the TMB assay kit (Calbiochem). Measurements were performed in the laboratory of Dr. S. Baldus (University Hospital Hamburg-Eppendorf)

These results indicate that H—NR can struggle the MPO-dependent deleterious effects in human endothelial cells.

FIG. 9. Effect of H—NR-1 on the binding of myeloperoxidase (MPO) to human umbilical vein endothelial cells (HUVEC). HUVEC were pre-incubated with myeloperoxidase (MPO; 1 μg/ml) for 1 hr; then non-bound MPO was washed out and cells were further incubated in the presence or absence of H—NR-1 (50 μM) for additional 30 min. After the incubation period, cells were washed out, lysed and MPO-protein content was determined by ELISA (Calbiochem). Measurements were performed in the laboratory of Dr. S. Baldus (University Hospital Hamburg-Eppendorf)

These results indicate that H—NR not only can directly scavenge the MPO-derived oxidants, but also replace the bound MPO from endothelium.

FIG. 10. In vivo EPR evidence for prolonged life time of H—NR. Anesthetized mice were injected with 1 mM H—NR-3 (0.5 ml-i.p.). The mouse tail was fixed in the resonator of a X-band EPR spectrometer (MS 200 Magnettech) and the spectra EPR were sequentially recorded.

It is likely that both H—NR circulating with blood and H—NR bound to vascular wall are responsible for these EPR spectra. Most importantly, the half-life of TEMPOL in vivo is known to last only a few minutes, while the half-life of H—NR according to the invention in vivo lasts several hours. These results substantiate the potential utilization of H—NR as perspective agents for in vivo EPR imaging.

FIG. 11. EPR (L-band) evidence for the prolonged life-time of H—NR in anesthetized mice (i.p. injection of 0.5 ml-1 mM solution of H—NR-3). The surface coil-type resonator (loop diameter 10 mm) was placed on the proximal part of mouse tail and the spectra EPR were recorded using an L-band (1.2 GHz) spectrometer RadicalScope mt 500 L (Magnettech GmbH, Berlin). Instrument parameters were 25 mW microwave power, 0.125 mT amplitude modulation, 100 kHz modulation frequency, center field 47.5 mT, sweep field 10 mT and 60 s sweep time.

This experiments shows that H—NR may be efficiently used as imaging agent in vivo. As such it serves as an potential contrasting agent for EPR imaging of vascular structures. In addition, the H—NR of the invention may be used for treating vascular diseases and for the preservation of vascular transplants.

FIG. 12. Dextran is unable to replace Heparin-nitroxide from the heparin-binding sites of the vascular wall. Rat aortic rings (3 mm long) were incubated (37° C.) with 100 μM H—NR-2 for 1 hr and then washed out with Krebs solution. Rings were incubated with H—NR-2 along (A) or in the presence of 1 mM dextran (B), or in the presence of 10 mM dextran (C). To estimate the amount of H—NR-2 bound to tissue, aortic rings were placed in capillaries, and spectra EPR(X-band) were recorded.

Thus, in contract to non-modified heparin (see FIG. 4), dextran is unable to replace heparin-nitroxide constructs from the heparin-binding sites of the vascular wall. This result demonstrates that the previously described dextran-nitroxide cannot target the same sites as the heparin-nitroxide agent of the invention.

FIGS. 13 and 14. Effect of heparin-nitroxide on the T1(T2)-enhanced magnetic resonance image of isolated pig blood vessels. Isolated pig aorta, carotids and coronaries were pre-treated with H—NR2 (1 mM; 30 min), then washed out in Krebs solution, placed in agar (0.8%) and (after 2 h) scanned using Trio MR tomograph (3 tesla).

The persistent enhancement of the both T1 and T2 MR contrast was observed in intimal and adventitial layer of the H—NR-treated vessels. These results provide evidence for the perspective use of heparin-nitroxides as MR contrast agents for imaging of the vascular wall.

1. Heparin-nitroxide derivative, comprising heparin and at least two and more nitroxides or polynitroxides that are covalently coupled to heparin by derivatisation of glycosaminoglycan carboxyl groups and/or glycosaminoglycan amino groups of heparin, the heparin-nitroxide derivative comprising the following general structure: 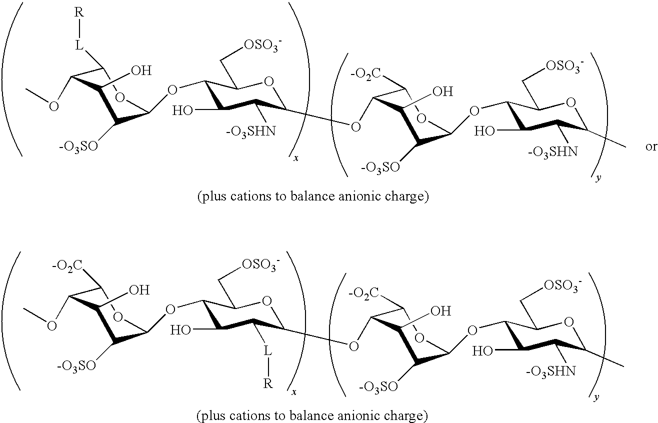
wherein R is a nitroxide/nitroxyl radical and L is an optional linker, wherein x,y>0.

2. The heparin-nitroxide derivative of claim 1, wherein the linker L comprises natural amino acids or short peptides residues, amide bond —C(O)NR′— or diamide bonds separated by hydrocarbon chain —C(O)NR′—(CH₂)ₘ—C(O)NR′— (CH₂)ₙ— or —C(O)NR′—(CH₂)ₘ—NR′C(O)—(CH₂)ₙ— or by any other suitable linker, R′ is a hydrogen or an alkyl substituent, wherein m, n≦0.

3. The heparin-nitroxide derivative of claim 2, wherein n is an integer from 0 to 2, and m is an integer from 1 to 6.

4. The heparin-nitroxide derivative of any of the preceding claims, wherein the nitroxide is a 5- or 6-atom N-heterocycle selected from the group consisting of piperidine, tetrahydropyridine, pyrroline, pyrrolidine, imidazoline, imidazolidine or oxazolidine.

5. The heparin-nitroxide derivative of any of the preceding claims, wherein more than 50% of the possible carboxyl and/or amino group binding sites at the glycoaminoglycan backbone of heparin are occupied by nitroxides/nitroxyl radicals.

6. The heparin-nitroxide derivative of any of the preceding claims, wherein the nitroxide is TEMPO (2,2,6,6-tetramethylpiperidine-1-oxyl).

7. The heparin-nitroxide derivative of any of the preceding claims, wherein more than 70% of the disaccharides of heparin are modified by TEMPO (2,2,6,6-tetramethylpiperidine-1-oxyl).

8. The heparin-nitroxide derivative of any of the preceding claims, wherein the agent comprises one of the following structures (I)-(IV): 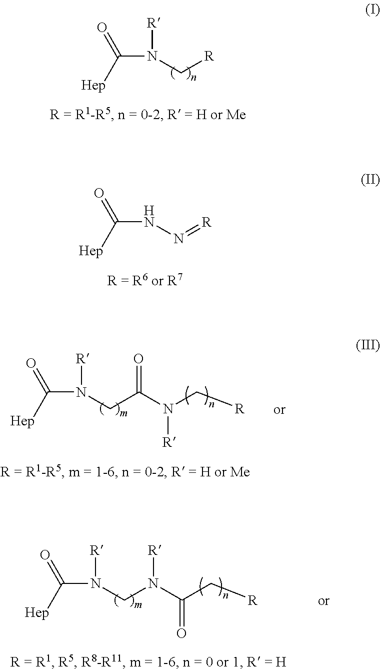
wherein R═R¹⁻¹¹ may be one of the following residues:
R¹=2,2,6,6-tetramethyl-1-oxylpiperidin-4-yl,
R²=3-amino-2,2,6,6-tetramethyl-1-oxylpiperidin-4-yl,
R³=4-alkyloxycarbonyl-2,2,6,6-tetramethyl-1-oxylpiperidin-4-yl,
R⁴=4-hydroxyimino-2,2,6,6-tetramethyl-1-oxylpiperidin-3-yl,
R⁵=2,2,5,5-tetramethyl-1-oxylpyrrolidin-3-yl,
R⁶=2,2,6,6-tetramethyl-1-oxylpiperidin-4-diyl,
R⁷=2,2,5,5-tetramethyl-1-oxylpyrrolidin-3-diyl,
R⁸=2,2,6,6-tetramethyl-1-oxyl-1,2,5,6-tetrahydropyridin-4-yl,
R⁹=4-acetylamino-2,2,6,6-tetramethyl-1-oxylpiperidin-4-yl,
R¹⁰°=2,2,5,5-tetramethyl-1-oxylpyrrolin-3-yl,
R¹¹=2,2,5,5-tetramethyl-4-bromo-1-oxylpyrrolin-3-yl.

9. The heparin-nitroxide derivative of any of the preceding claims, wherein the agent comprises one of the following structures (I)-(III): 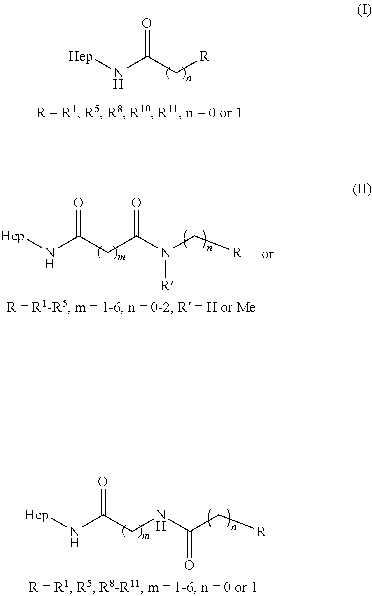
wherein R═R¹⁻¹¹ may be one of the following residues:
R¹=2,2,6,6-tetramethyl-1-oxylpiperidin-4-yl,
R²=3-amino-2,2,6,6-tetramethyl-1-oxylpiperidin-4-yl,
R³=4-alkyloxycarbonyl-2,2,6,6-tetramethyl-1-oxylpiperidin-4-yl,
R⁴=4-hydroxyimino-2,2,6,6-tetramethyl-1-oxylpiperidin-3-yl,
R⁵=2,2,5,5-tetramethyl-1-oxylpyrrolidin-3-yl,
R⁶=2,2,6,6-tetramethyl-1-oxylpiperidin-4-diyl,
R⁷=2,2,5,5-tetramethyl-1-oxylpyrrolidin-3-diyl,
R⁸=2,2,6,6-tetramethyl-1-oxyl-1,2,5,6-tetrahydropyridin-4-yl,
R⁹=4-acetylamino-2,2,6,6-tetramethyl-1-oxylpiperidin-4-yl,
R¹⁰=2,2,5,5-tetramethyl-1-oxylpyrrolin-3-yl,
R¹¹=2,2,5,5-tetramethyl-4-bromo-1-oxylpyrrolin-3-yl.

10. The heparin-nitroxide derivative of any of the preceding claims, wherein heparin has a molecular weight of approximately between 5 and 40 kDa.

11. A method for producing the heparin-nitroxide derivatives of any one of claims 1 to 10, wherein the method comprises the step of:
(a) coupling of nitroxides/polynitroxide radicals to heparin by coupling of amino, carboxyl, carboxylic acid anhydride or isocyano groups of nitroxide to glycosaminoglycan carboxyl or amino groups of heparin.

12. The method of claim 11, wherein the derivatisation is performed under anhydrous conditions.

13. The method of claim 11 or 12, wherein for the coupling reaction the NHS/R—NH₂ molar ratios are within the range from 1:10 to 1:1.

14. The method of claims 11 to 13, wherein carbodiimide is added to the mixture of heparin/NHS/amino-nitroxide rather than the other way round.

15. A method of using a heparin-nitroxide derivative of any one of claims 1 to 10 for specifically targeting nitroxide to the endothelial cell surface and/or extracellular matrix (ECM) of vascular tissue.

16. A method of using a heparin-nitroxide derivative of any one of claims 1 to 10 for electron paramagnetic resonance imaging (EPRI) or magnetic resonance imaging (MRI) of the intimal layer of biological vessels.

17. A method for electron paramagnetic resonance imaging (EPRI) of vascular structure in biological vessels, in particular the vascular intima of conductive blood vessels, comprising:
(a) contacting vascular tissue with a paramagnetic heparin-nitroxide derivative of any one of claims 1 to 10,
(b) washing of unbound heparin-nitroxide derivative,
(c) measuring of EPR signals obtained from vascular-bound paramagnetic heparin-nitroxide derivative.

18. A method of using of a heparin-nitroxide derivative of any one of claims 1 to 10 for preventing or diminishing oxidative stress on the endothelial cell surface and/or extracellular matrix (ECM) of vascular tissue.

19. A method of using a heparin-nitroxide derivative of any one of claims 1 to 10 for the preservation of biological transplants.

20. A pharmaceutical composition, comprising a heparin-nitroxide derivative of any one of claims 1 to 10, and a pharmaceutically acceptable carrier.

21. A composition comprising a heparin-nitroxide derivative of any one of claims 1 to 10 for use in a method for treatment of acute and chronic diseases associated with oxidative extracellular stress.

22. The composition of claim 21, wherein the disease is selected from the group consisting of oxidative stress-dependent platelet activation, cardiovascular disease, neurodegenerative diseases such as Alzheimer's, pulmonary disease, thrombosis, chronic inflammatory disease, diabetes, ischemia, rheumatoid arthritis, cardiac infarct, cancer, hypertension, ocular damage, ischemia-reperfusion injury, and septic shock.

23. Antioxidant agent for therapeutic purposes, comprising a heparin-nitroxide derivative of any one of claims 1 to 10.

24. Paramagnetic probe for diagnostic purposes, comprising a heparin-nitroxide derivative of any one of claims 1 to 10.
